(19) United States
(12) Patent Application Publication
(10) Pub. No.: US 2011/0039895 A1
(43) Pub. Date: Feb. 17, 2011

(54) PROLYL HYDROXYLASE INHIBITORS

(75) Inventors: Deping Chai, Collegoville, PA (US); Duke M. Fitch, King of Prussia, PA (US)

Correspondence Address: GlaxoSmithKline;GLOBAL PATENTS -US, UW2220, P. O. BOX 1539, KING OF PRUSSIA, PA, 19406-0939, US

(73) Assignee: Glaxo Smith Kline LLC., a corporation

(21) Appl. No.: 12/989,487

(22) PCT Filed: Apr. 29, 2009
(86) PCT No.: PCT/US2009/042054

§ 371 Date: Oct. 25, 2010

Related U.S. Application Data
(60) Provisional application No. 61/049,071, filed on Apr. 30, 2008.

(51) Int. Cl. A61K 31/4439; C07D 235/06; C07D 235/18; C07D 235/08; C07D 401/10; C07D 235/24; A61K 31/4184; A61P 7/06

(52) U.S. Cl. 514/338; 548/304.4; 548/310.7; 546/273.4; 548/309.4; 514/394

(57) ABSTRACT

The invention described herein relates to certain benzimidazol-4-ylcarboxamide derivatives of formula (I)

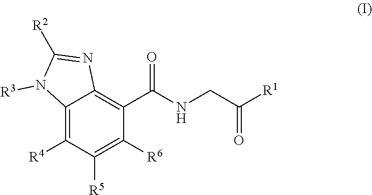

which are antagonists of HIF prolyl hydroxylases and are useful for treating diseases benefiting from the inhibition of this enzyme, anemia being one example.

### RELATED APPLICATION DATA

This application claims priority from U.S. Provisional Application No. 61/049,066, filed 30 Apr. 2008, the contents of which are incorporated herein by reference.

### FIELD OF THE INVENTION

This invention relates to certain benzimidazol-4-ylcarboxamide derivatives that are inhibitors of HIF prolyl hydroxylases, and thus have use in treating diseases benefiting from the inhibition of this enzyme, anemia being one example.

### BACKGROUND OF THE INVENTION

Anemia occurs when there is a decrease or abnormality in red blood cells, which leads to reduced oxygen levels in the blood. Anemia occurs often in cancer patients, particularly those receiving chemotherapy. Anemia is often seen in the elderly population, patients with renal disease, and in a wide variety of conditions associated with chronic disease.

Frequently, the cause of anemia is reduced erythropoietin (Epo) production resulting in prevention of erythropoiesis (maturation of red blood cells). Epo production can be increased by inhibition of prolyl hydroxylases that regulate hypoxia inducible factor (HIF).

One strategy to increase erythropoietin (Epo) production is to stabilize and thus increase the transcriptional activity of the HIF. HIF-alpha subunits (HIF-1 alpha, HIF-2alpha, and HIF-3alpha) are rapidly degraded by proteosome under normoxic conditions upon hydroxylation of proline residues by prolyl hydroxylases (EGLN1, 2, 3). Proline hydroxylation allows interaction with the von Hippel Lindau (VHL) protein, a component of an E3 ubiquitin ligase. This leads to ubiquitination of HIF-alpha and subsequent degradation. Under hypoxic conditions, the inhibitory activity of the prolyl hydroxylases is suppressed, HIF-alpha subunits are therefore stabilized, and HIF-responsive genes, including Epo, are transcribed. Thus, inhibition of prolyl hydroxylases results in increased levels of HIF-alpha and thus increased Epo production.

The compounds of this invention provide a means for inhibiting these hydroxylases, increasing Epo production, and thereby treating anemia. Ischemia, stroke, and cytoprotection may also benefit by administering these compounds.

### SUMMARY OF THE INVENTION

In the first instance, this invention relates to a compound of formula (I):

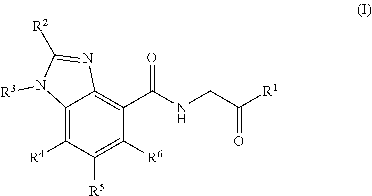

wherein:

R¹ is —NR⁷R⁸ or —OR⁹;

R² is selected from the group consisting of hydrogen, nitro, cyano, —C(O)R¹², —C(O)OR¹², —OR¹², —SR¹², —S(O)R¹², —S(O)₂R¹²—CONR¹⁰R¹¹, —N(R¹⁰)C(O)R¹², —N(R¹⁰)C(O)OR¹², —OC(O)NR¹⁰R¹¹, —N(R¹⁰)C(O)N¹⁰R¹¹, —SO₂NR¹⁰R¹¹, —N(R¹⁰SO₂R¹², C₁-C₁₀ alkyl, C₂-C₁₀ alkenyl, C₂-C₁₀ alkynyl, C₃-C₈ cycloalkyl, C₃-C₈ heterocycloalkyl, C₅-C₈ cycloalkenyl, aryl, and heteroaryl;

R³ is selected from the group consisting of hydrogen, —C(O)R¹², —C(O)OR¹², —S(O)₂R¹², —CONR¹⁰R¹¹, —SO₂NR¹⁰R¹¹, C₁-C₁₀ alkyl, C₂-C₁₀ alkenyl, C₂-C₁₀ alkynyl, C₃-C₈ cycloalkyl, C₃-C₈ heterocycloalkyl, C₅-C₈ cycloalkenyl, aryl, and heteroaryl;

R⁴, R⁵, and R⁶ are each independently selected from the group consisting of hydrogen, nitro, cyano, halogen, —C(O)R¹², —C(O)OR¹², —OR¹², —SR¹², —S(O)R¹², —S(O)₂R¹², —NR¹⁰R¹¹, —CONR¹⁰R¹¹, —N(R¹⁰)C(O)R¹², —N(R¹⁰)C(O)OR¹², —OC(O)NR¹⁰R¹¹, —N(R¹⁰)C(O)N¹⁰R¹¹, —P(O)(OR¹²)₂, —SO₂NR¹⁰R¹¹, —N(R¹⁰SO₂R¹², C₁-C₁₀ alkyl, C₁-C₁₀ alkenyl, C₁-C₁₀ alkynyl, C₃-C₈ cycloalkyl, C₃-C₈ heterocycloalkyl, C₅-C₈ cycloalkenyl, aryl, and heteroaryl;

R⁷ and R⁸ are each independently selected from the group consisting of hydrogen, C₁-C₆ alkyl, C₂-C₆ alkenyl, C₂-C₆ alkynyl, C₃-C₆ cycloalkyl, C₃-C₆ heterocycloalkyl, aryl, and heteroaryl;

R⁹ is hydrogen, or a cation, or C₁-C₄ alkyl;

R¹⁰ and R¹¹ are each independently selected from the group consisting of hydrogen, C₁-C₁₀ alkyl, C₃-C₈ cycloalkyl, C₁-C₁₀ alkyl-C₃-C₈ cycloalkyl, C₃-C₈ heterocycloalkyl, C₁-C₁₀ alkyl C₃-C₈ heterocycloalkyl, aryl, C₁-C₁₀ alkyl-aryl, heteroaryl, C₁-C₁₀ alkyl-heteroaryl, —CO(C₁-C₄ alkyl), —CO(C₃-C₆ cycloalkyl), —CO(C₃-C₆ heterocycloalkyl), —CO(aryl), —CO(heteroaryl), and —SO₂(C₁-C₄ alkyl); or R¹⁰ and R¹¹ taken together with the nitrogen to which they are attached form a 5- or 6- or 7-membered saturated ring optionally containing one other heteroatom which is oxygen, nitrogen or sulfur;

each R¹² is independently selected from the group consisting of hydrogen, C₁-C₁₀ alkyl, C₂-C₁₀ alkenyl, C₂-C₁₀ alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈ heterocycloalkyl, aryl, C₁-C₁₀ alkyl-aryl, heteroaryl, and C₁-C₁₀ alkyl-heteroaryl;
any carbon or heteroatom of R², R³, R⁴, R⁵, R⁶, R⁷, R⁸, R⁹, R¹¹ or R¹² is unsubstituted or, where possible, is substituted with one or more substituents independently selected from C₁-C₆ alkyl, aryl, heteroaryl, halogen, —OR cyano, nitro, —C(O)R¹², —C(O)OR¹², —SR¹², —S(O)R¹², —S(O)₂R¹², —N(R¹⁰)C(O)R¹², —N(R¹⁰)C(O)OR¹², —OC(O)NR¹⁰R¹¹, —N(R¹⁰)C(O)NR¹⁰R¹¹, —SO₂NR¹⁰R¹¹, —N(R¹⁰)SO₂R¹², C₂-C₁₀ alkenyl, C₂-C₁₀ alkynyl, C₃-C₈ cycloalkyl, C₃-C₈ heterocycloalkyl, C₅-C₈ cycloalkenyl, aryl or heteroaryl, wherein R¹⁰, R¹¹, and R¹² are the same as defined above;

or a pharmaceutically acceptable salt or solvate thereof.

In a second aspect of the present invention, there is provided a compound of formula (I) or a salt or solvate thereof for use in mammalian therapy, e.g. treating anemia. An example of this therapeutic approach is that of a method for treating anemia caused by increasing the production of erythropoietin (Epo) by inhibiting HIF prolyl hydroxylases comprising administering a compound of formula (I) to a patient in need thereof, neat or admixed with a pharmaceutically acceptable excipient, in an amount sufficient to increase production of Epo.

In a third aspect of the present invention, there is provided a pharmaceutical composition comprising a compound of formula (I) or a salt, solvate, or the like thereof, and one or more of pharmaceutically acceptable carriers, diluents and excipients.

In a fourth aspect, there is provided the use of a compound of formula (I) or a salt or solvate thereof in the preparation of a medicament for use in the treatment of a disorder mediated by inhibiting HIF prolyl hydroxylases, such as an anemia, that can be treated by inhibiting HIF prolyl hydroxylases.

### DETAILED DESCRIPTION OF THE INVENTION

For the avoidance of doubt, unless otherwise indicated, the term “substituted” means substituted by one or more defined groups. In the case where groups may be selected from a number of alternative groups the selected groups may be the same or different.

The term “independently” means that where more than one substituent is selected from a number of possible substituents, those substituents may be the same or different.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a straight- or branched-chain hydrocarbon radical having the specified number of carbon atoms, so for example, as used herein, the terms “C₁-C₄ alkyl” and “C₁-C₁₀ alkyl” refers to an alkyl group having at least 1 and up to 4 or 10 carbon atoms respectively. Examples of such branched or straight-chained alkyl groups useful in the present invention include, but are not limited to, methyl, ethyl, n-propyl, isopropyl, isobutyl, n-butyl, t-butyl, n-pentyl, isopentyl, n-hexyl, n-heptyl, n-octyl, n-nonyl, and n-decyl, and branched analogs of the latter 5 normal alkanes.

When the term “alkenyl” (or “alkenylene”) is used it refers to straight or branched hydrocarbon chains containing the specified number of carbon atoms and at least 1 and up to 5 carbon-carbon double bonds. Examples include ethenyl (or ethenylene) and propenyl (or propenylene).

When the term “alkynyl” (or “alkynylene”) is used it refers to straight or branched hydrocarbon chains containing the specified number of carbon atoms and at least 1 and up to 5 carbon-carbon triple bonds. Examples include ethynyl (or ethynylene) and propynyl (or propynylene).

When “cycloalkyl” is used it refers to a non-aromatic, saturated, cyclic hydrocarbon ring containing the specified number of carbon atoms. So, for example, the term “C₃-C₈ cycloalkyl” refers to a non-aromatic cyclic hydrocarbon ring having from three to eight carbon atoms. Exemplary “C₃-C₈ cycloalkyl” groups useful in the present invention include, but are not limited to, cyclopropyl, cyclobutyl, cyclopentyl, cyclohexyl, cycloheptyl and cyclooctyl.

The term “C₅-C₈cycloalkenyl” refers to a non-aromatic monocyclic carboxycyclic ring having the specified number of carbon atoms and up to 3 carbon-carbon double bonds. “Cycloalkenyl” includes by way of example cyclopentenyl and cyclohexenyl.

Where “C₃-C₈ heterocycloalkyl” is used, it means a non-aromatic heterocyclic ring containing the specified number of ring atoms being, saturated or having one or more degrees of unsaturation and containing one or more heteroatom substitutions selected from O, S and/or N. Such a ring may be optionally fused to one or more other “heterocyclic” ring(s) or cycloalkyl ring(s). Examples of “heterocyclic” moieties include, but are not limited to, aziridine, thiirane, oxirane, azetidine, oxetane, thietane, tetrahydrofuran, pyran, 1,4-dioxane, 1,4-dithiane, 1,3-dioxane, 1,3-dioxolane, piperidine, piperazine, 2,4-piperazinedione, pyrrolidine, 2-imidazoline, imidazolidine, pyrazolidine, pyrazoline, morpholine, thiomorpholine, tetrahydrothiopyran, tetrahydrothiophene, and the like.

“Aryl” refers to optionally substituted monocyclic and polycarbocyclic unfused or fused groups having 6 to 14 carbon atoms and having at least one aromatic ring that complies with Hackers Rule. Examples of aryl groups are phenyl, biphenyl, naphthyl, anthracenyl, phenanthrenyl and the like.

“Heteroaryl” means an optionally substituted aromatic monocyclic ring or polycarbocyclic fused ring system wherein at least one ring complies with Hiickel's Rule, has the specified number of ring atoms, and that ring contains at least one heteroatom selected from N, O, and/or S. Examples of “heteroaryl” groups include furanyl, thienyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridinyl, pyridazinyl, pyrazinyl, pyrimidinyl, quinolinyl, isoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, benzofuranyl, benzothienyl, benzimidazolyl, benzthiazolyl, indolizinyl, indolyl, isoindolyl, and indazolyl.

The term “optionally” means that the subsequently described event(s) may or may not occur, and includes both event(s), which occur, and events that do not occur.

The term “solvate” refers to a complex of variable stoichiometry formed by a solute and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Herein, the term “pharmaceutically-acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

In certain embodiments, compounds according to Formula I may contain an acidic functional group, one acidic enough to form salts. Representative salts include pharmaceutically-acceptable metal salts such as sodium, potassium, lithium, calcium, magnesium, aluminum, and zinc salts; carbonates and bicarbonates of a pharmaceutically-acceptable metal cation such as sodium, potassium, lithium, calcium, magnesium, aluminum, and zinc; pharmaceutically-acceptable organic primary, secondary, and tertiary amines including aliphatic amines, aromatic amines, aliphatic diamines, and hydroxy alkylamines such as methylamine, ethylamine, 2-hydroxyethylamine, diethylamine, triethylamine, ethylenediamine, ethanolamine, diethanolamine, and cyclohexylamine.

In certain embodiments, compounds according to Formula (I) may contain a basic functional group and are therefore capable of forming pharmaceutically-acceptable acid addition salts by treatment with a suitable acid. Suitable acids include pharmaceutically-acceptable inorganic acids and pharmaceutically-acceptable organic acids. Representative pharmaceutically-acceptable acid addition salts include hydrochloride, hydrobromide, nitrate, methylnitrate, sulfate, bisulfate, sulfamate, phosphate acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate.

Compounds of particular interest include those wherein:

R¹ is —NR⁷R⁸ or —OR⁹;

R² is selected from the group consisting of hydrogen, cyano, —C(O)R¹², —C(O)OR¹², —S(O)₂R¹²—NR¹⁰R¹¹, —CONR¹⁰R¹¹, —N(R¹⁰)C(O)R¹², —N(R¹⁰)C(O)N¹⁰R¹¹, —N(R¹⁰SO₂R¹², C₁-C₁₀ alkyl, C₂-C₁₀ alkenyl, C₂-C₁₀ alkynyl, C₃-C₈ cycloalkyl, C₃-C₈ heterocycloalkyl, C₅-C₈ cycloalkenyl, aryl, and heteroaryl;

R³ is selected from the group consisting of hydrogen, —C(O)R¹², —C(O)OR¹², —S(O)₂R¹², —CONR¹⁰R¹¹, —SO₂NR¹⁰R¹¹, C₁-C₁₀ alkyl, C₂-C₁₀ alkenyl, C₂-C₁₀ alkynyl, C₃-C₈ cycloalkyl, C₃-C₈ heterocycloalkyl, C₅-C₈ cycloalkenyl, aryl, and heteroaryl;

R⁴, R⁵, and R⁶ are each independently selected from the group consisting of hydrogen, cyano, halogen, —C(O)R¹², —C(O)OR¹², —OR¹², —NR¹⁰R¹¹, —CONR¹⁰R¹¹, —N(R¹⁰)C(O)R¹², —N(R¹⁰)C(O)N¹⁰R¹¹, C₁-C₁₀ alkyl, C₂-C₁₀ alkenyl, C₂-C₁₀ alkynyl, C₃-C₈ cycloalkyl, C₃-C₈ heterocycloalkyl, C₅-C₈ cycloalkenyl, aryl, and heteroaryl;

R⁷ and R⁸ are each independently selected from the group consisting of hydrogen, C₁-C₆ alkyl, C₂-C₆ alkenyl, C₂-C₆ alkynyl, C₃-C₆ cycloalkyl, C₃-C₆ heterocycloalkyl, aryl, and heteroaryl;

R⁹ is hydrogen, or a cation, or C₁-C₄ alkyl;

R¹⁰ and R¹¹ are each independently selected from the group consisting of hydrogen, C₁-C₆ alkyl, C₃-C₆ cycloalkyl, C₃-C₆ heterocycloalkyl, aryl, heteroaryl, —CO(C₁-C₄ alkyl), —CO(C₃-C₆ cycloalkyl), —CO(C₃-C₆ heterocycloalkyl), —CO(aryl), —CO(heteroaryl), and —SO₂(C₁-C₄ alkyl); or

R¹⁰ and R¹¹ taken together with the nitrogen to which they are attached form a 5- or 6- or 7-membered saturated ring optionally containing one other heteroatom which is oxygen, nitrogen or sulfur;

each R¹² is independently selected from the group consisting of hydrogen, C₁-C₆ alkyl, C₂-C₆alkenyl, C₂-C₆ alkynyl, —CO(C₁-C₄ alkyl), —CO(aryl), —CO(heteroaryl), —CO(C₃-C₆ cycloalkyl), —CO(C₃-C₆heterocycloalkyl), C₃-C₆ cycloalkyl, C₃-C₆ heterocycloalkyl, aryl, and heteroaryl;

any carbon or heteroatom of R², R³, R⁴, R⁵, R⁶, R⁷, R⁸, R⁹, R¹⁰, R¹¹ or R¹² is unsubstituted or, where possible, is substituted with one or more substituents independently selected from C₁-C₆ alkyl, aryl, heteroaryl, halogen, —OR¹², —NR¹⁰R¹¹, cyano, —C(O)R¹², —C(O)OR¹², —CONR¹⁰R¹¹, —N(R¹⁰)C(O)R¹², —N(R¹⁰)C(O)OR¹², —OC(O)NR¹⁰R¹¹, —OC(O)NR¹⁰R¹¹, —SO₂NR¹⁰R¹¹, —N(R¹⁰SO₂R¹², C₂-C₆ alkenyl, C₂-C₆ alkynyl, C₃-C₆ cycloalkyl, C₃-C₆ heterocycloalkyl, C₅-C₈ cycloalkenyl, aryl, or heteroaryl, wherein R¹⁰, R¹¹, and R¹² are the same as defined above;

Compounds of further interest are those wherein:

R¹ is —OR⁹;

R² is selected from the group consisting of hydrogen, cyano, —C(O)R¹², —C(O)OR¹², —CONR¹⁰R¹¹, C₁-C₆ alkyl, C₂-C₆ alkenyl, C₂-C₆ alkynyl, C₃-C₆ cycloalkyl, C₃-C₆ heterocycloalkyl, aryl, and heteroaryl;

R³ is selected from the group consisting of hydrogen, —S(O)₂R¹², C₁-C₁₀ alkyl, C₂-C₁₀ alkenyl, C₂-C₁₀ alkynyl, C₃-C₈ cycloalkyl, C₃-C₈ heterocycloalkyl, C₅-C₈ cycloalkenyl, aryl, and heteroaryl;

R⁴, R⁵, and R⁶ are each independently selected from the group consisting of hydrogen, cyano, halogen, —OR¹², —NR¹⁰R¹¹, —CONR¹⁰R¹¹, C₁-C₆ alkyl, C₃-C₆cycloalkyl, C₃-C₆ heterocycloalkyl, aryl, and heteroaryl;

R⁹ is hydrogen, or a cation;

R¹⁰ and R¹¹ are each independently selected from the group consisting of hydrogen, C₁-C₆ alkyl, C₃-C₆ cycloalkyl, C₃-C₆ heterocycloalkyl, aryl, heteroaryl, —CO(C₁-C₄ alkyl), —CO(C₃-C₆ cycloalkyl), —CO(C₃-C₆ heterocycloalkyl), —CO(aryl), —CO(heteroaryl), and —SO₂(C₁-C₄ alkyl); or

R¹⁰ and R¹¹ taken together with the nitrogen to which they are attached form a 5- or 6- or 7-membered saturated ring optionally containing one other heteroatom which is oxygen, nitrogen or sulfur;

each R¹² is independently selected from the group consisting of hydrogen, C₁-C₆ alkyl, C₂-C₆alkenyl, C₂-C₆ alkynyl, —CO(C₁-C₄ alkyl), —CO(aryl), —CO(heteroaryl), —CO(C₃-C₆ cycloalkyl), —CO(C₃-C₆heterocycloalkyl), C₃-C₆ cycloalkyl, C₃-C₆ heterocycloalkyl, aryl, and heteroaryl;

any carbon or heteroatom of R², R³, R⁴, R⁵, R⁶, R⁹, R¹⁰, R¹¹ or R¹² is unsubstituted or where possible, is substituted with one or more substituents independently selected from C₁-C₆ alkyl, aryl, heteroaryl, halogen, —OR¹², —NR¹⁰R¹¹, cyano, —C(O)R¹², —C(O)OR¹², —CONR¹⁰R¹¹, —N(R¹⁰)C(O)R¹², —N(R¹⁰)C(O)OR¹², —OC(O)NR¹⁰R¹¹, —N(R¹⁰)C(O)NR¹⁰R¹¹, —SO₂NR¹⁰R¹¹, —N(R¹⁰)SO₂R¹², C₂-C₆ alkenyl, C₂-C₆ alkynyl, C₃-C₆ cycloalkyl, C₃-C₆ heterocycloalkyl, C₅-C₈ cycloalkenyl, aryl, or heteroaryl, wherein R¹⁰, R¹¹ and R¹² are the same as defined above;

Of further interest are those compounds where:

R¹ is —OR⁹;

R² is selected from the group consisting of hydrogen, cyano, —C(O)NR¹⁰R¹¹, C₁-C₆ alkyl, C₃-C₆ cycloalkyl, C₃-C₆ heterocycloalkyl, aryl, and heteroaryl;

R³ is selected from the group consisting of hydrogen, C₁-C₆ alkyl, C₃-C₆ cycloalkyl, C₃-C₆ heterocycloalkyl, C₅-C₆ cycloalkenyl, aryl, and heteroaryl;

R⁴ is hydrogen;

R⁵ and R⁶ are each independently selected from the group consisting of hydrogen, cyano, halogen, —OR¹², —NR¹⁰R¹¹, —CONR¹⁰R¹¹, C₁-C₆ alkyl, C₃-C₆cycloalkyl, C₃-C₆heterocycloalkyl, aryl, and heteroaryl;

R⁹ is hydrogen, or a cation;

R¹⁰ and R¹¹ are each independently selected from the group consisting of hydrogen, C₁-C₆ alkyl, C₃-C₆ cycloalkyl, C₃-C₆ heterocycloalkyl, aryl, and heteroaryl; or R¹⁰ and R¹¹ taken together with the nitrogen to which they are attached form a 5- or 6- or 7-membered saturated ring optionally containing one other heteroatom which is oxygen, nitrogen or sulfur;

each R¹² is independently selected from the group consisting of hydrogen, C₁-C₆ alkyl, C₃-C₆ cycloalkyl, C₃-C₆ heterocycloalkyl, aryl, and heteroaryl;

any carbon or heteroatom of R², R³, R⁵, R⁶, R¹⁰, R¹¹ or R¹² is unsubstituted or, where possible, is substituted with one or more substituents independently selected from C₁-C₆ alkyl, aryl, heteroaryl, halogen, —OR¹², —NR¹⁰R¹¹, cyano, —C(O)R¹², —C(O)OR¹², —CONR¹⁰R¹¹, —N(R¹⁰)C(O)R¹², —N(R¹⁰)C(O)OR¹², —OC(O)NR¹⁰R¹¹, —NR¹⁰)C(O)NR¹⁰R¹¹, —SO₂NR¹⁰R¹¹, —N(R¹⁰)SO₂R¹², C₂-C₆alkenyl, C₂-C₆ alkynyl, C₃-C₆ cycloalkyl, C₃-C₆ heterocycloalkyl, C₅-C₈ cycloalkenyl, aryl, or heteroaryl, wherein R¹⁰ and R¹² are the same as defined above;

Specific compounds exemplified herein are:
1) N-{[5-hydroxy-1-(phenylmethyl)-1H-benzimidazol-4-yl]carbonyl}glycine;
2) N-{[5-(methyloxy)-1-(phenylmethyl)-1H-benzimidazol-4-yl]carbonyl}glycine;
3) N-({5-[(phenylmethyl)oxy]-1H-benzimidazol-4-yl}carbonyl)glycine;
4) N-[(5-hydr oxy-1H-benzimidazol-4-yl)carbonyl]glycine;
5) N-{[1-[(2-chlorophenyl)methyl]-5-(methyloxy)-1H-benzimidazol-4-yl]carbonyl}glycine;
6) N-({1-[(2-chlorophenyl)methyl]-5-hydr oxy-1H-benzimidazol-4-yl}carbonyl)glycine;
7) N-{[5-fluoro-1-(phenylmethyl)-1H-benzimidazol-4-yl]carbonyl}glycine;
8) N-[(5-fluoro-1H-benzimidazol-4-yl)carbonyl]glycine;
9) N-{[1-(phenylmethyl)-1H-benzimidazol-4-yl]carbonyl}glycine;
10) N-(1H-benzimidazol-4-ylcarbonyl)glycine;
11) N-{[5-(methyloxy)-2-phenyl-1-(phenylmethyl)-1H-benzimidazol-4-yl]carbonyl}glycine;
12) N-{[2-methyl-5-(methyloxy)-1-(phenylmethyl)-1H-benzimidazol-4-yl]carbonyl}glycine;
13) N-{[5-(methylamino)-1H-benzimidazol-4-yl]carbonyl}glycine;
14) N-{[6-bromo-5-(methyloxy)-1-(phenylmethyl)-1H-benzimidazol-4-yl]carbonyl}glycine;
15) N-{[6-bromo-5-hydroxy-1-(phenylmethyl)-1H-benzimidazol-4-yl]carbonyl}glycine;
16) N-[(5-ethyl-1H-benzimidazol-4-yl)carbonyl]glycine;
17) N-{[5-(dimethylamino)-1-(phenylmethyl)-1H-benzimidazol-4-yl]carbonyl}glycine;
18) N-{[5-(methyloxy)-1H-benzimidazol-4-yl]carbonyl}glycine;
19) N-{[1-[(4-bromophenyl)methyl]-5-(methyloxy)-1H-benzimidazol-4-yl]carbonyl}glycine;
20) N-{[1-(4-biphenylylmethyl)-5-(methyloxy)-1H-benzimidazol-4-yl]carbonyl}glycine;
21) N-[(5-(methyloxy)-1-{[4-(4-pyridinyl)phenyl]methyl}-1H-benzimidazol-4-yl)carbonyl]glycine;
22) N-[(5-(methyloxy)-1-{[4-(3-pyridinyl)phenyl]methyl}-1H-benzimidazol-4-yl)carbonyl]glycine; and
23) N-{[2-(aminocarbonyl)-5-(methyloxy)-1-(phenylmethyl)-1H-benzimidazol-4-yl]carbonyl}glycine.

Processes for preparing the compound of formula (I) are also within the ambit of this invention. To illustrate, process for preparing a compound of formula (I)

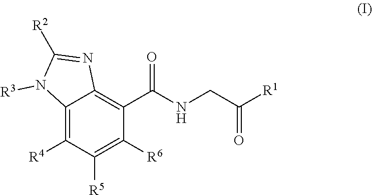

wherein R¹, R², R³, R⁴, R⁵, and R⁶ are the same as defined above for formula (I), the process comprising treating a compound of formula A:

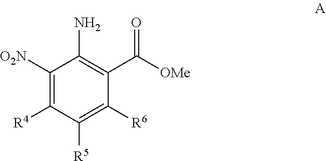

wherein R⁴, R⁵, and R⁶ are the same as for those groups in formula (I), in a hydrogen atmosphere with an appropriate catalyst, such as palladium on charcoal, in an appropriate solvent, such as ethyl acetate, followed by addition of an appropriately substituted orthoester, such as trimethyl orthoformate, neat or in an appropriate solvent, such as methanol, along with an appropriate acid, such as anhydrous hydrochloric acid in 1,4-dioxane or diethyl ether, to form a compound of formula B:

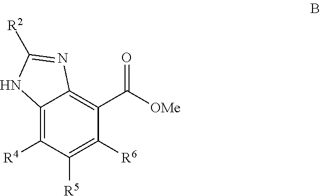

wherein R², R⁴, R⁵, and R⁶ are the same as for those groups in formula (I), which may then be deprotonated with an appropriate base, such as sodium hydride, in an appropriate solvent, such as tetrahydrofuran or N,N-dimethylformamide, and reacted with an appropriate alkylating agent, such as benzyl bromide or 2-chlorobenzyl bromide, followed by ester hydrolysis with an appropriate base, such as sodium hydroxide, in an appropriate solvent, such as tetrahydrofuran/methanol, to form a compound of formula C:

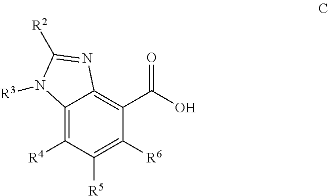

wherein R², R³, R⁴, R⁵, and R⁶ are the same as for those groups in formula (I), which is then coupled with an appropriate glycine ester, such as glycine ethyl ester hydrochloride, and an appropriate base, such as triethylamine or diisopropylethylamine, and an appropriate coupling reagent, such as HATU or PyBOP, in an appropriate solvent, such as N,N-dimethylformamide, followed by ester hydrolysis with an appropriate base, such as sodium hydroxide, in an appropriate solvent, such as tetrahydrofuran/methanol, to form a compound of formula (I) where R¹ is —OH.

The compounds of formula (I) may be prepared in crystalline or non-crystalline form, and, if crystalline, may optionally be solvated, e.g. as the hydrate. This invention includes within its scope stoichiometric solvates (e.g. hydrates) as well as compounds containing variable amounts of solvent (e.g. water).

Certain of the compounds described herein may contain one or more chiral atoms, or may otherwise be capable of existing as two enantiomers. The compounds claimed below include mixtures of enantiomers as well as purified enantiomers or enantiomerically enriched mixtures. Also included within the scope of the invention are the individual isomers of the compounds represented by formula (I), or claimed below, as well as any wholly or partially equilibrated mixtures thereof. The present invention also covers the individual isomers of the claimed compounds as mixtures with isomers thereof in which one or more chiral centers are inverted. Also, it is understood that any tautomers and mixtures of tautomers of the claimed compounds are included within the scope of the compounds of formula (I) as disclosed herein above or claimed herein below.

Where there are different isomeric forms they may be separated or resolved one from the other by conventional methods, or any given isomer may be obtained by conventional synthetic methods or by stereospecific or asymmetric syntheses.

While it is possible that, for use in therapy, a compound of formula (I), as well as salts, solvates and the like, may be administered as a neat preparation, i.e. no additional carrier, the more usual practice is to present the active ingredient confected with a carrier or diluent. Accordingly, the invention further provides pharmaceutical compositions, which includes a compound of formula (I) and salts, solvates and the like, and one or more pharmaceutically acceptable carriers, diluents, or excipients. The compounds of formula (I) and salts, solvates, etc, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I), or salts, solvates etc, with one or more pharmaceutically acceptable carriers, diluents or excipients.

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolized in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Preferred prodrugs for compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals.

Pharmaceutical compositions may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula (I), depending on the condition being treated, the route of administration and the age, weight and condition of the patient, or pharmaceutical compositions may be presented in unit dose forms containing a predetermined amount of active ingredient per unit dose. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association a compound of formal (I) with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of a compound of formula (I). Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit pharmaceutical compositions for oral administration can be microencapsulated. The formulation can also be prepared to prolong or sustain the release as for example by coating or embedding particulate material in polymers, wax or the like.

Pharmaceutical compositions adapted for rectal administration may be presented as suppositories or as enemas.

Pharmaceutical compositions adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The pharmaceutical compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the pharmaceutical compositions may include other agents conventional in the art having regard to the type of formulation in question, for example those suitable for oral administration may include flavoring agents.

A therapeutically effective amount of a compound of the present invention will depend upon a number of factors including, for example, the age and weight of the intended recipient, the precise condition requiring treatment and its severity, the nature of the formulation, and the route of administration, and will ultimately be at the discretion of the attendant prescribing the medication. However, an effective amount of a compound of formula (I) for the treatment of anemia will generally be in the range of 0.1 to 100 mg/kg body weight of recipient per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, etc., may be determined as a proportion of the effective amount of the compound of formula (I) per se. It is envisaged that similar dosages would be appropriate for treatment of the other conditions referred to above.

### DEFINITIONS

h—hour(s)
min.—minute(s)
MgSO₄—Magnesium sulfate,
Na₂SO₄—Sodium sulfate,
NaH—Sodium hydride,
NaOH—Sodium hydroxide,
Pd/C—Palladium on charcoal,
PyBOP—Benzotriazol-1-yl-oxytripyrrolidinophosphonium hexafluorophosphate,
HATU—2-(1H-7-azabenzotriazol-1-yl)-1,1,3,3-tetramethyluronium hexafluorophosphate.

### Chemical Background:

The compounds of this invention may be made by a variety of methods, including standard chemistry. Any previously defined variable will continue to have the previously defined meaning unless otherwise indicated. Illustrative general synthetic methods are set out below and then specific compounds of the invention as prepared are given in the examples.

Compounds of general formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognize if a stereocenter exists in compounds of formula (I). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

The compounds described herein may be made from commercially available starting materials or synthesized using known organic, inorganic and/or enzymatic processes.

### Illustrated Methods of Preparation

### Schemes

Included in the present invention is a process according to Scheme 1 for the synthesis of the compounds:

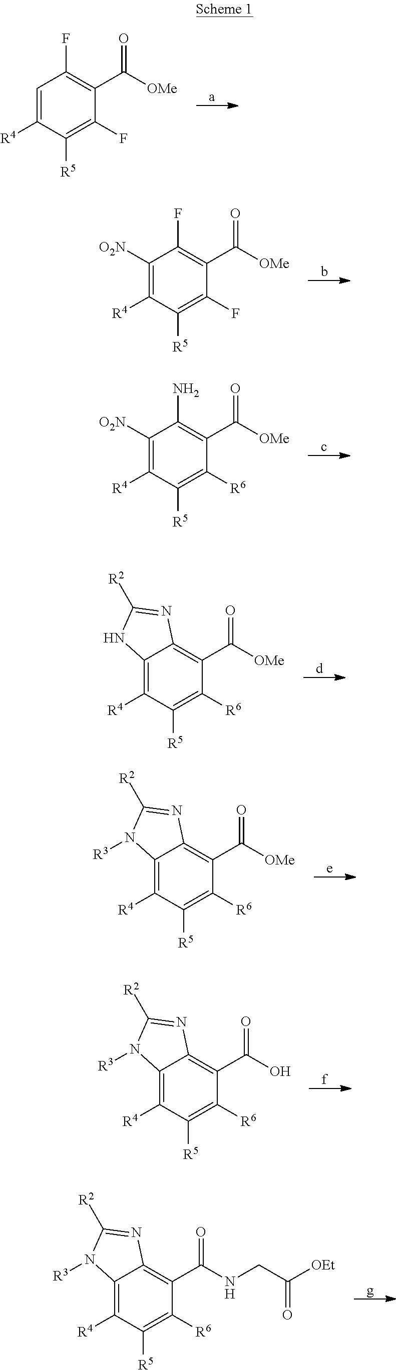

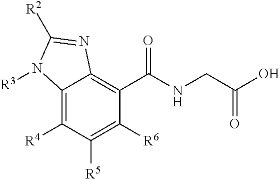

a) fuming nitric acid, concentrated sulfuric acid; b) ammonium hydroxide, ethanol, then R⁶Na or R⁶H, methanol or tetrahydrofuran; c) H₂, Pd/C, ethyl acetate, or i. bromine, dichloromethane, ii. H₂, Pd/C, ethyl acetate, then R²C(OCH₃)₃, HCl in 1,4-dioxane, methanol; d) NaH, R³Br, tetrahydrofuran or N,N-dimethylformamide; e) NaOH, tetrahydrofuran/methanol; f) glycine ethyl ester hydrochloride, triethylamine or diisopropylethylamine, HATU or PyBOP, N,N-dimethylformamide; g) NaOH, tetrahydrofuran/methanol.

### Example 1

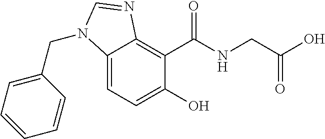

### N-{[5-hydroxy-1-(phenylmethyl)-1H-benzimidazol-4-yl]carbonyl}glycine

### 1a) Methyl 2-amino-6-fluoro-3-nitrobenzoate

To fuming nitric acid (3.87 mL, 86.6 mmol) at 0° C. was slowly added concentrated sulfuric acid (7.27 mL, 136.4 mmol). After stirring for 5 min., methyl 2,6-difluorobenzoate (3.90 mL, 29.0 mmol) was added and the reaction mixture was allowed to warm to ambient temperature. After 30 min, the reaction mixture was poured into ice-water, and extracted thrice with dichloromethane. The combined organic portions were washed with saturated aqueous sodium bicarbonate, dried over MgSO₄, filtered, and concentrated in vacuo to afford a colorless oil. MS (ES+) m/e 218 [M+H]⁺. Upon standing, the oil solidified to a white solid, which was dissolved in ethanol (50.0 mL) and treated with ammonium hydroxide (1.0 mL, 29% aqueous solution) at ambient temperature. After 4 h, additional ammonium hydroxide (0.8 mL, 29% aqueous solution) was added and the reaction mixture was stirred overnight. The solution was concentrated and the residual solid was washed with isopropanol, filtered, washed with water, and dried in vacuo to afford the title compound (5.69 g, 92%) as a yellow solid. ¹H NMR (400 MHz, DMSO-d₆) δ ppm 8.33 (dd, J=9.5, 5.7 Hz, 1H), 8.11 (br. s., 2H), 6.62 (t, J=9.9 Hz, 1H), 3.89 (s, 3H). MS (ES+) m/e 215 [M+H]⁺.

### 1b) Methyl 2-amino-6-(methyloxy)-3-nitrobenzoate

To a solution of sodium methoxide (25% in MeOH)(12.8 mL, 56.0 mmol) in methanol (150 mL) at 0° C. was added methyl 2-amino-6-fluoro-3-nitrobenzoate (prepared as in Example 1a) (9.75 g, 45.5 mmol). Following removal of the ice bath, the reaction mixture was stirred at ambient temperature for 1 h and then quenched with 1N aqueous hydrochloric acid. The resulting precipitate was filtered, washed with water, and dried in vacuo to afford the title compound (8.06 g, 78%) as a yellow solid. ¹H NMR (400 MHz, DMSO-d₆) δ ppm 8.20 (d, J=9.6 Hz, 1H), 7.38 (br. s., 2H), 6.56 (d, J=9.6 Hz, 1H), 3.87 (s, 3H), 3.82 (s, 3H). MS (ES+) m/e 227 [M+H]⁺.

### 1c) Methyl 5-(methyloxy)-1H-benzimidazole-4-carboxylate

To a solution of methyl 2-amino-6-(methyloxy)-3-nitrobenzoate (prepared as in Example 1b) (1.00 g, 4.42 mmol) in ethyl acetate (75.0 mL) was added 10% palladium on charcoal (0.100 g, 0.094 mmol), followed by evacuation of the reaction vessel and purging with nitrogen. The reduction was carried out under 40 psi of hydrogen gas with a Parr Shaker overnight. The reaction mixture was filtered through Celite®, washed through with ethyl acetate, and concentrated in vacuo. To the residue were added trimethyl orthoformate (3.00 mL, 29.1 mmol) and ytterbium trifluoromethanesulfonate (0.014 g, 0.022 mmol), followed by heating to 90° C. for 3 h. Upon cooling, the mixture was purified via flash column chromatography (10% methanol in methylene chloride) to afford the title compound (0.690 g, 75%) as a yellow solid. ¹H NMR (400 MHz, DMSO-d₆) δ ppm 12.3 (br. s., 1H), 8.14 (s, 1H), 7.82 (br. s., 1H), 7.06 (d, J=9.1 Hz, 1H), 3.90 (s, 3H), 3.87 (s, 3H), 3.86 (s, 3H). MS (ES+) m/e 207 [M+H]⁺.

### 1d) Methyl 5-(methyloxy)-1-(phenylmethyl)-1H-benzimidazole-4-carboxylate

To a solution of the compound from Example 1c) (0.690 g, 3.30 mmol) in N,N-dimethylformamide (5.0 mL) was added sodium hydride (60% dispersion in mineral oil) (0.150 g, 3.75 mmol). After the mixture was stirred at ambient temperature for 5 min., benzyl bromide (0.400 mL, 3.40 mmol) was added. The reaction was stirred at ambient temperature for 3 h, quenched with ice water, and extracted with ethyl acetate. The organic extract was dried over MgSO₄, filtered, concentrated in vacuo, and purified via flash column chromatography (10% methanol in methylene chloride) to afford the title compound (0.340 g, 35%) as a yellow solid. ¹H NMR (400 MHz, CHLOROFORM-d) δ ppm 8.01 (s, 1H), 7.32-7.39 (m, 3H), 7.27 (d, J=8.8 Hz, 2H), 7.13-7.20 (m, 2H), 6.96 (d, J=8.8 Hz, 1H), 5.35 (s, 2H), 4.07 (s, 3H), 3.91 (s, 3H). MS (ES+) m/e 297 [M+H]⁺.

### 1e) 5-Hydroxy-1-(phenylmethyl)-1H-benzimidazole-4-carboxylic acid

A solution of methyl 5-(methyloxy)-1-(phenylmethyl)-1H-benzimidazole-4-carboxylate (prepared as in Example 1d) (0.200 g, 0.670 mmol) in 48% aqueous hydrobromic acid (5.0 mL) was heated at 100° C. for 1 hour. Upon cooling, the precipitate was collected by filtration, washed with acetone, and dried in vacuo to afford the title compound (0.120 g, 67%) as a grey solid. MS (ES+) m/e 269 [M+H]⁺.

### 1f) Ethyl N-{[5-hydroxy-1-(phenylmethyl)-1H-benzimidazol-4-yl]carbonyl}glycinate

To a solution of the compound from Example 1e) (0.090 g, 0.320 mmol) in N,N-dimethylformamide (10.0 mL) were added triethylamine (0.140 mL, 0.960 mmol) and HATU (0.265 g, 0.700 mmol). Glycine ethyl ester hydrochloride (0.089 g, 0.64 mmol) was added and the mixture was stirred overnight at ambient temperature, quenched with water, and extracted with methylene chloride. The organic layer was washed with 1N aqueous hydrochloric acid, dried over MgSO₄, filtered, concentrated in vacuo, and purified via flash column chromatography (10% methanol in methylene chloride) to afford the title compound (0.050 g, 48%) as a yellow solid. ¹H NMR (400 MHz, CHLOROFORM-d) δ ppm 12.8 (s, 1H), 10.5 (br. s., 1H), 8.00 (s, 1H), 7.34-7.43 (m, 3H), 7.30 (d, J=8.8 Hz, 1H), 7.15-7.22 (m, 2H), 6.94 (d, J=8.8 Hz, 1H), 5.38 (s, 2H), 4.35 (d, J=3.8 Hz, 2H), 4.30 (q, J=7.2 Hz, 2H), 1.34 (t, J=7.2 Hz, 3H). MS (ES+) m/e 354 [M+H]⁺.

### 1g) N-{[5-hydroxy-1-(phenylmethyl)-1H-benzimidazol-4-yl]carbonyl}glycine

To a solution of the compound from Example 1f) (0.050 g, 0.150 mmol) in tetrahydrofuran (5.0 mL) was added 6N aqueous sodium hydroxide (0.500 mL, 3.00 mmol). The reaction was stirred overnight at ambient temperature, followed by purification via preparative HPLC (YMC 75×30 mm column, 0.1% TFA in water and 0.1% TFA in acetonitrile) to afford the TFA salt of the title compound (0.015 g, 31%) as a white solid. ¹H NMR (400 MHz, MeOD) δ ppm 9.01 (s, 1H), 7.66 (d, J=9.1 Hz, 1H), 7.31-7.48 (m, 5H), 7.08 (d, J=8.8 Hz, 1H), 5.64 (s, 2H), 4.27 (s, 2H). MS (ES+) m/e 326 [M+H]⁺.

### Example 2

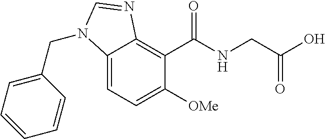

### N-{[5-(methyloxy)-1-(phenylmethyl)-1H-benzimidazol-4-yl]carbonyl}glycine

### 2a) 5-(Methyloxy)-1-(phenylmethyl)-1H-benzimidazole-4-carboxylic acid

To a solution of methyl 5-(methyloxy)-1-(phenylmethyl)-1H-benzimidazole-4-carboxylate (prepared as in Example 1d) (0.460 g, 1.54 mmol) in methanol (5.0 mL) was added 6N aqueous sodium hydroxide (1.00 mL, 6.00 mmol). The mixture was heated to 50° C. for 5 h, allowed to cool to ambient temperature, neutralized with 1N aqueous hydrochloric acid, and extracted with ethyl acetate. The extract was dried over MgSO₄, filtered, and concentrated in vacuo to afford the title compound (0.340 g, 75%) as a white solid. ¹H NMR (400 MHz, DMSO-d₆) δ ppm 13.0 (br. s., 1H), 8.46 (s, 1H), 7.56 (d, J=9.1 Hz, 1H), 7.23-7.38 (m, 5H), 7.07 (d, J=9.1 Hz, 1H), 5.50 (s, 2H), 3.80 (s, 3H). MS (ES+) m/e 283 [M+H]⁺.

### 2b) Ethyl N-{[5-(methyloxy)-1-(phenylmethyl)-1H-benzimidazol-4-yl]carbonyl}glycine

To a solution of the compound from Example 2a) (0.070 g, 0.250 mmol) in N,N-dimethylformamide (10.0 mL) were added triethylamine (0.100 mL, 0.750 mmol) and HATU (0.104 g, 0.270 mmol). Glycine ethyl ester hydrochloride (0.070 g, 0.500 mmol) was added and the mixture was stirred overnight at ambient temperature. The reaction mixture was concentrated in vacuo and the resulting yellow oil was dissolved in methanol (5.0 mL). 6N aqueous sodium hydroxide (0.500 mL, 3.00 mmol) was added and the mixture was stirred overnight at ambient temperature. Purification via preparative HPLC (YMC 75×30 mm column, 0.1% TFA in water and 0.1% TFA in acetonitrile) provided the TFA salt of the title compound (0.012 g, 22%) as a white solid. ¹H NMR (400 MHz, MeOD) δ ppm 9.20 (s, 1H), 7.84 (d, J=8.3 Hz, 1H), 7.31-7.55 (m, 6H), 5.70 (s, 2H), 4.22 (s, 3H). MS (ES+) m/e 368 [M+H]⁺.

### Example 3

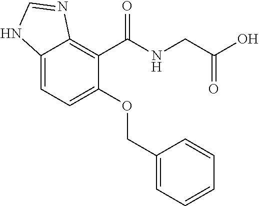

### N-({5-[(phenylmethyl)oxy]-1H-benzimidazol-4-yl}carbonyl)glycine

### 3a) Phenylmethyl 2-amino-3-nitro-6-[(phenylmethyl)oxy]benzoate

To benzyl alcohol (5.0 mL) was added sodium hydride (60% dispersion in mineral oil) (0.373 g, 9.30 mmol). After the gas evolution ceased, the compound from Example 2a) (1.00 g, 4.67 mmol) was added. The mixture was stirred at ambient temperature for 3 h. The reaction was quenched with water, and extracted with ethyl acetate. The extract was dried over MgSO₄, concentrated in vacuo, and purified via preparative HPLC (YMC 75×30 mm column, 0.1% TFA in water and 0.1% TFA in acetonitrile) to afford the title compound (1.20 g, 68%) as a yellow solid. ¹H NMR (400 MHz, DMSO-d₆) δ ppm 8.19 (d, J=9.6 Hz, 1H), 7.47 (br. s., 2H), 7.25-7.39 (m, 10 H), 6.65 (d, J=9.9 Hz, 1H), 5.31 (s, 2H), 5.27 (s, 2H). MS (ES+) m/e 379 [M+H]⁺.

### 3b) Phenylmethyl 5-[(phenylmethyl)oxy]-1H-benzimidazole-4-carboxylate

To a solution of the compound from Example 3a) (1.20 g, 3.20 mmol) in water (1.0 mL), acetonitrile (30.0 mL) and ethanol (30.0 mL) was added tin(II) chloride dihydrate (7.20 g, 32.0 mmol). After stirring at reflux overnight, the reaction was quenched by saturated aqueous sodium carbonate and extracted with methylene chloride. The extract was dried over MgSO₄, filtered, and concentrated in vacuo. To the residue were added trimethyl orthoformate (5.00 mL, 48.5 mmol) and ytterbium trifluoromethanesulfonate (0.0035 g, 0.0056 mmol), followed by heating to 90° C. for 3 h. Upon cooling, the reaction was concentrated in vacuo and purified via flash column chromatography (10% methanol in methylene chloride) to afford the title compound (0.744 g, 65%) as a yellow solid. MS (ES+) m/e 359 [M+H]⁺.

### 3c) 5-[(Phenylmethyl)oxy]-1H-benzimidazole-4-carboxylic acid

To a solution of the compound from Example 3b) (0.500 g, 1.33 mmol) in methanol (5.00 mL) was added 6N aqueous sodium hydroxide (0.500 mL, 3.00 mmol). The mixture was heated to reflux for 3 h, allowed to cool to ambient temperature, neutralized with 1N aqueous hydrochloric acid, and extracted with ethyl acetate. The extract was dried over MgSO₄, filtered, and concentrated in vacuo to afford the title compound (0.231 g, 65%) as a yellow solid. ¹H NMR (400 MHz, DMSO-d₆) δ ppm 8.14 (s, 1H), 7.78 (d, J=8.8 Hz, 1H), 7.54 (d, J=7.3 Hz, 2H), 7.28-7.34 (m, 1H), 7.13 (d, J=8.8 Hz, 1H), 5.25 (s, 2H). MS (ES+) m/e 269 [M+H]⁺.

### 3d) N-({5-[(phenylmethyl)oxy]-1H-benzimidazol-4-yl}carbonyl)glycine

Following the procedure of Example 2b), except substituting the compound from Example 3c) for the compound from Example 2a), the title compound was obtained as a white solid. ¹H NMR (400 MHz, MeOD) δ ppm 9.14 (s, 1H), 7.92 (d, J=9.1 Hz, 1H), 7.57 (dd, J=6.1, 2.3 Hz, 3H), 7.29-7.47 (m, 3H), 5.55 (s, 2H), 4.23 (s, 2H). MS (ES+) m/e 326 [M+H]⁺.

### Example 4

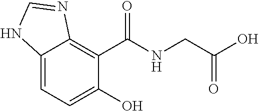

### N-[(5-hydr oxy-1H-benzimidazol-4-yl)carbonyl]glycine

To a solution of the compound from Example 3d) (0.050 g, 0.150 mmol) in methanol (50.0 mL) was added 10% palladium on charcoal (0.005 g, 0.005 mmol), followed by evacuation of the reaction vessel and purging with nitrogen. The reduction was carried out under 40 psi of hydrogen gas with a Parr Shaker overnight. The reaction mixture was filtered through Celite® and purified via preparative HPLC (YMC 75×30 mm column, 0.1% TFA in water and 0.1% TFA in acetonitrile) to afford the TFA salt of the title compound (0.030 g, 79%) as a brown solid. ¹H NMR (400 MHz, MeOD) δ ppm 9.21 (s, 1H), 7.84 (d, J=8.8 Hz, 1H), 7.29 (d, J=8.8 Hz, 1H), 4.27 (s, 2H). MS (ES+) m/e 236 [M+H]⁺.

### Example 5

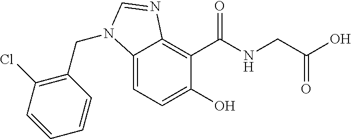

### N-({1-[(2-chlorophenyl)methyl]-5-hydroxy-1H-benzimidazol-4-yl}carbonyl)glycine

### 5a) Methyl 1-[(2-chlorophenyl)methyl]-5-(methyloxy)-1H-benzimidazole-4-carboxylate

To a solution of methyl 5-(methyloxy)-1H-benzimidazole-4-carboxylate (prepared as in Example 1c) (0.760 g, 3.70 mmol) in N,N-dimethylformamide (10.0 mL) was added sodium hydride (60% dispersion in mineral oil) (0.177 g, 4.40 mmol). After the mixture was stirred at ambient temperature for 5 min., 2-chlorobenzyl bromide (0.440 mL, 3.70 mmol) was added. The reaction was stirred at ambient temperature for 3 h, quenched with ice water, and extracted with ethyl acetate. The organic extract was dried over MgSO₄, filtered, concentrated in vacuo, and purified via preparative HPLC (YMC 75×30 mm column, 0.1% TFA in water and 0.1% TFA in acetonitrile) to afford the TFA salt of the title compound (0.300 g, 25%) as a white solid. ¹H NMR (400 MHz, DMSO-d₆) δ ppm 9.17 (s, 1H), 7.82 (d, J=9.1 Hz, 1H), 7.56 (dd, J=7.8, 1.3 Hz, 1H), 7.41 (td, J=7.7, 1.8 Hz, 1H), 7.35 (dd, J=7.8, 1.3 Hz, 1H), 7.32 (d, J=7.7 Hz, 1H), 7.19 (dd, J=7.7, 1.6 Hz, 1H), 5.76 (s, 2H), 3.90 (s, 3H), 3.88 (s, 3H). MS (ES+) m/e 331 [M+H]⁺.

### 5b) 1-[(2-Chlorophenyl)methyl]-5-hydroxy-1H-benzimidazole-4-carboxylic acid

A solution of the compound from Example 5a) (0.700 g, 2.12 mmol) in 48% aqueous hydrobromic acid (10.0 mL) was heated at 100° C. for 30 min. Upon cooling, the reaction was quenched by water. The resulting precipitate was collected by filtration and purified via preparative HPLC (YMC 75×30 mm column, 0.1% TFA in water and 0.1% TFA in acetonitrile) to afford the TFA salt of the title compound (0.190 g, 28%) as a white solid. MS (ES+) m/e 303 [M+H]⁺.

### 5c) N-({1-[(2-chlorophenyl)methyl]-5-hydroxy-1H-benzimidazol-4-yl}carbonyl)glycine

To a solution of the compound from Example 5b) (0.190 g, 0.630 mmol) in N,N-dimethylformamide (10.0 mL) were added triethylamine (0.540 mL, 3.80 mmol) and HATU (0.660 g, 2.76 mmol). Glycine ethyl ester hydrochloride (0.352 g, 2.52 mmol) was added and the mixture was stirred overnight at ambient temperature. The reaction mixture was concentrated in vacuo and the resulting yellow oil was dissolved in methanol (5.0 mL). 6N aqueous sodium hydroxide (0.500 mL, 3.00 mmol) was added and the mixture was stirred overnight at ambient temperature. Purification via preparative HPLC (YMC 75×30 mm column, 0.1% TFA in water and 0.1% TFA in acetonitrile) provided the TFA salt of the title compound (0.042 g, 19%) as a white solid. ¹H NMR (400 MHz, MeOD) δ ppm 8.83 (br. s., 1H), 7.65 (d, J=9.1 Hz, 1H), 7.53 (dd, J=7.8, 1.0 Hz, 1H), 7.41 (td, J=7.6, 1.6 Hz, 1H), 7.35 (td, J=7.5, 1.1 Hz, 1H), 7.28 (d, J=8.8 Hz, 1H), 7.07 (d, J=8.8 Hz, 1H), 5.74 (s, 2H), 4.28 (s, 2H). MS (ES+) m/e 360 [M+H]⁺.

### Example 6

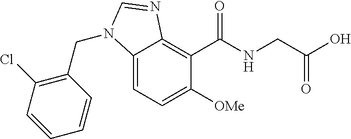

### N-{[1-[(2-chlorophenyl)methyl]-5-(methyloxy)-1H-benzimidazol-4-yl]carbonyl}glycine

### 6a) 1-[(2-Chlorophenyl)methyl]-5-(methyloxy)-1H-benzimidazole-4-carboxylic acid

A solution of the compound from Example 5a) (0.700 g, 2.12 mmol) in 48% aqueous hydrobromic acid (10.0 mL) was heated at 100° C. for 30 min. Upon cooling, the reaction was quenched by water. The resulting precipitate was collected by filtration and purified via preparative HPLC (YMC 75×30 mm column, 0.1% TFA in water and 0.1% TFA in acetonitrile) to afford the TFA salt of the title compound (0.040 g, 6%) as a white solid. MS (ES+) m/e 317 [M+H]⁺.

### 6b) N-{[1-[(2-chlorophenyl)methyl]-5-(methyloxy)-1H-benzimidazol-4-yl]carbonyl}glycine

Following the procedure of Example 5c), except substituting the compound from Example 6a) for the compound from Example 5b), the TFA salt of the title compound was obtained as a white solid. ¹H NMR (400 MHz, MeOD) δ ppm 9.47 (s, 1H), 7.97 (d, J=9.4 Hz, 1H), 7.54-7.61 (m, 2H), 7.46-7.51 (m, 2H), 7.39-7.45 (m, 1H), 5.90 (s, 2H), 4.17 (s, 3H). MS (ES+) m/e 346 [M+H]⁺.

### Example 7

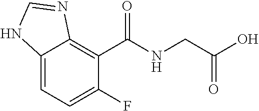

### N-[(5-fluoro-1H-benzimidazol-4-yl)carbonyl]glycine

### 7a) Methyl 5-fluoro-1H-benzimidazole-4-carboxylate

Following the procedure of Example 1c), except substituting methyl 2-amino-6-fluoro-3-nitrobenzoate (prepared as in Example 1a) for methyl 2-amino-6-(methyloxy)-3-nitrobenzoate, the title compound was obtained as a white solid. ¹H NMR (400 MHz, DMSO-d₆) δ ppm 12.6 (br. s., 1H), 8.31 (s, 1H), 7.94 (dd, J=8.6, 4.3 Hz, 1H), 7.17 (dd, J=12.0, 8.7 Hz, 1H), 3.94 (s, 3H). MS (ES+) m/e 209 [M+H]⁺.

### 7b) N-[(5-fluoro-1H-benzimidazol-4-yl)carbonyl]glycine

To a solution of the compound from Example 7a) (0.300 g, 1.50 mmol) in ethanol (3.0 mL) were added 1,8-diazabicyclo[5.4.0]undec-7-ene (0.700 mL, 3.00 mmol) and glycine (0.232 g, 3.00 mmol), followed by heating to 150° C. for 30 min in a Biotage Initiator® microwave synthesizer. Upon cooling, the reaction was purified via preparative HPLC (YMC 75×30 mm column, 0.1% TFA in water and 0.1% TFA in acetonitrile) to afford the TFA salt of the title compound (0.019 g, 5.3%) as a white solid. ¹H NMR (400 MHz, DMSO-d₆) δ ppm 12.8 (br. s., 1H), 9.02 (br. s., 1H), 8.48 (s, 1H), 7.83 (dd, J=8.8, 4.3 Hz, 1H), 7.24 (dd, J=12.1, 8.8 Hz, 1H), 4.06 (d, J=5.8 Hz, 2H). MS (ES+) m/e 238 [M+H]⁺.

### Example 8

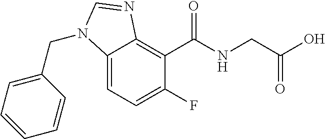

### N-{[5-fluoro-1-(phenylmethyl)-1H-benzimidazol-4-yl]carbonyl}glycine

### 8a) Methyl 5-fluoro-1-(phenylmethyl)-1H-benzimidazole-4-carboxylate

To a solution of the compound from Example 7a) (0.600 g, 3.10 mmol) in N,N-dimethylformamide (15.0 mL) was added sodium hydride (60% dispersion in mineral oil) (0.248 g, 6.20 mmol). After the mixture was stirred at ambient temperature for 5 min., benzyl bromide (0.370 mL, 3.10 mmol) was added. The reaction was stirred at ambient temperature for 2 h, quenched with ice water, and extracted with ethyl acetate. The organic extract was dried over MgSO₄, filtered, concentrated in vacuo, and purified via preparative HPLC (YMC 75×30 mm column, 0.1% TFA in water and 0.1% TFA in acetonitrile) to afford the TFA salt of the title compound (0.540 g, 53%) as a white solid. MS (ES+) m/e 285 [M+H]⁺.

### 8b) N-{[5-fluoro-1-(phenylmethyl)-1H-benzimidazol-4-yl]carbonyl}glycine

Following the procedure of Example 7b), except substituting the compound from Example 8a) for the compound from Example 7a), the title compound was obtained as a yellow solid. ¹H NMR (400 MHz, MeOD) δ ppm 9.30 (s, 1H), 7.90 (dd, J=9.1, 4.0 Hz, 1H), 7.24-7.51 (m, 6H), 5.73 (s, 2H), 4.24 (s, 2H). MS (ES+) m/e 328 [M+H]⁺.

### Example 9

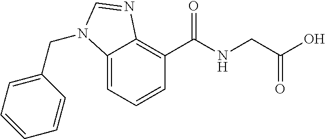

### N-{[1-(phenylmethyl)-1H-benzimidazol-4-yl]carbonyl}glycine

### 9a) methyl 1-(phenylmethyl)-1H-benzimidazole-4-carboxylate

Following the procedure of Example 1d), except substituting methyl 1H-benzimidazole-4-carboxylate for the compound from Example 1c), the title compound was obtained as a yellow solid. MS (ES+) m/e 267 [M+H]⁺.

### 9b) N-{[1-(phenylmethyl)-1H-benzimidazol-4-yl]carbonyl}glycine

Following the procedure of Example 7b), except substituting the compound from Example 9a) for the compound from Example 7a), the title compound was obtained as a white solid. ¹H NMR (400 MHz, MeOD) δ ppm 8.42 (s, 1H), 8.00 (d, J=6.8 Hz, 1H), 7.66 (d, J=8.1 Hz, 1H), 7.24-7.42 (m, 6H), 5.58 (s, 2H), 4.31 (s, 2H). MS (ES+) m/e 310 [M+H]⁺.

### Example 10

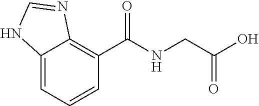

### N-(1H-benzimidazol-4-ylcarbonyl)glycine

Following the procedure of Example 7b), except substituting methyl 1H-benzimidazole-4-carboxylate for the compound from Example 7a), the TFA salt of the title compound was obtained as a yellow solid. ¹H NMR (400 MHz, MeOD) δ ppm 9.29 (s, 1H), 8.10 (d, J=7.6 Hz, 1H), 8.02 (d, J=8.3 Hz, 1H), 7.70 (t, J=8.0 Hz, 1H), 4.22 (s, 2H). MS (ES+) m/e 220 [M+H]⁺.

### Example 11

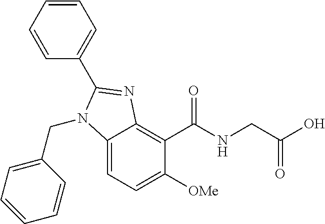

### N-{[5-(methyloxy)-2-phenyl-1-(phenylmethyl)-1H-benzimidazol-4-yl]carbonyl}glycine

### 11a) Methyl 2,3-diamino-6-(methyloxy)benzoate

To a solution of methyl 2-amino-6-(methyloxy)-3-nitrobenzoate (prepared as in Example 1b) (0.530 g, 2.34 mmol) in ethyl acetate (50.0 mL) was added 10% palladium on charcoal (0.125 g, 0.117 mmol), followed by evacuation of the reaction vessel and purging with nitrogen. The reduction was carried out under 50 psi of hydrogen gas with a Parr Shaker overnight. The reaction mixture was filtered through Celite®, washed through with ethyl acetate, and concentrated in vacuo to afford the title compound (0.460 g, 100%) as a dark, viscous oil. The material was used without further purification. ¹H NMR (400 MHz, CHLOROFORM-d) ppm 6.76 (d, J=8.3 Hz, 1H), 6.19 (d, J=8.6 Hz, 1H), 3.91 (s, 3H), 3.78 (s, 3H). MS (ES+) m/e 197 [M+H]⁺.

### 11b) Methyl 5-(methyloxy)-2-phenyl-1H-benzimidazole-4-carboxylate

To an aqueous solution of sodium bisulfite (40% in water, 15.0 mL, 4.40 mmol) were added methyl 2,3-diamino-6-(methyloxy)benzoate (prepared as in Example 11a) (1.00 g, 3.10 mmol) and a solution of benzaldehyde (0.450 mL, 4.40 mmol) in ethanol (15.0 mL). The reaction mixture was heated to reflux for 4 h, allowed to cool to ambient temperature, and extracted with ethyl acetate. The extract was dried over MgSO₄, filtered, concentrated in vacuo and purified via flash column chromatography (0-100% ethyl acetate in hexanes) to afford the title compound (0.470 g, 38%) as a white solid. ¹H NMR (400 MHz, CHLOROFORM-d) δ ppm 10.9 (br. s., 1H), 8.07 (dd, J=8.1, 1.5 Hz, 2H), 7.94 (d, J=8.8 Hz, 1H), 7.45-7.57 (m, 3H), 7.00 (d, J=9.1 Hz, 1H), 4.05 (s, 3H), 4.02 (s, 3H). MS (ES+) m/e 283 [M+H]⁺.

### 11c) Methyl 5-(methyloxy)-2-phenyl-1-(phenylmethyl)-1H-benzimidazole-4-carboxylate

To a solution of the compound from Example 11b) (0.470 g, 1.60 mmol) in N,N-dimethylformamide (5.0 mL) at 0° C. was added sodium hydride (60% dispersion in mineral oil) (0.074 g, 1.83 mmol). After the mixture was stirred at 0° C. for 5 min., benzyl bromide (0.193 mL, 1.60 mmol) was added. Following removal of the cooling bath, the reaction was stirred overnight at ambient temperature, quenched with ice water, and extracted with ethyl acetate. The organic extract was dried over MgSO₄, filtered, concentrated in vacuo, and purified via flash column chromatography (0-100% ethyl acetate in hexanes) to afford the title compound (0.230 g, 40%) as a yellow solid. MS (ES+) m/e 373 [M+H]⁺.

### 11d) 5-(Methyloxy)-2-phenyl-1-(phenylmethyl)-1H-benzimidazole-4-carboxylic acid

To a solution of the compound from Example 11c) (0.100 g, 0.270 mmol) in methanol (10.0 mL) was added 6N aqueous sodium hydroxide (0.220 mL, 1.30 mmol). The mixture was heated to 50° C. overnight, allowed to cool to ambient temperature, neutralized with 1N aqueous hydrochloric acid, and extracted with ethyl acetate. The extract was concentrated in vacuo and purified via flash column chromatography (10% methanol in methylene chloride) to afford the title compound (0.028 g, 29%) as a yellow solid. MS (ES+) m/e 359 [M+H]⁺.

### 11e) N-{[5-(methyloxy)-2-phenyl-1-(phenylmethyl)-1H-benzimidazol-4-yl]carbonyl}glycine

Following the procedure of Example 2b), except substituting the compound from Example 11d) for the compound from Example 2a), the title compound was obtained as a white solid. ¹H NMR (400 MHz, DMSO-d₆) δ ppm 8.97 (t, J=5.7 Hz, 1H), 7.86 (d, J=9.1 Hz, 1H), 7.80 (d, J=7.1 Hz, 2H), 7.67 (d, J=7.3 Hz, 1H), 7.62 (d, J=7.6 Hz, 2H), 7.40 (d, J=9.1 Hz, 1H), 7.25-7.34 (m, 3H), 7.10 (d, J=6.1 Hz, 2H), 5.67 (s, 2H), 4.07 (d, J=5.8 Hz, 2H), 3.98 (s, 3H). MS (ES+) m/e 416 [M+H]⁺.

### Example 12

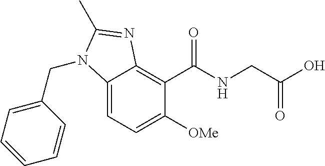

### N-{[2-methyl-5-(methyloxy)-1-(phenylmethyl)-1H-benzimidazol-4-yl]carbonyl}glycine

### 12a) Methyl 2-methyl-5-(methyloxy)-1H-benzimidazole-4-carboxylate

Following the procedure of Example 11b), except substituting acetaldehyde for benzaldehyde, the title compound was obtained as a yellow solid. ¹H NMR (400 MHz, CHLOROFORM-d) δ ppm 10.4 (br. s., 1H), 7.76 (d, J=8.6 Hz, 1H), 6.89 (d, J=8.8 Hz, 1H), 3.96 (s, 3H), 3.95 (s, 3H), 2.59 (s, 3H). MS (ES+) m/e 221 [M+H]⁺.

### 12b) Methyl 2-methyl-5-(methyloxy)-1-(phenylmethyl)-1H-benzimidazole-4-carboxylate

Following the procedure of Example 11c), except substituting the compound from Example 12a) for the compound from Example 11b), the title compound was obtained as a yellow solid. MS (ES+) m/e 311 [M+H]⁺.

### 12c) 2-Methyl-5-(methyloxy)-1-(phenylmethyl)-1H-benzimidazole-4-carboxylic acid

Following the procedure of Example 11d), except substituting the compound from Example 12b) for the compound from Example 11c), the title compound was obtained as a yellow solid. MS (ES+) m/e 297 [M+H]⁺.

### 12d) N-{[2-methyl-5-(methyloxy)-1-(phenylmethyl)-1H-benzimidazol-4-yl]carbonyl}glycine

Following the procedure of Example 2b), except substituting the compound from Example 12c) for the compound from Example 2a), the TFA salt of the title compound was obtained as a yellow solid. ¹H NMR (400 MHz, DMSO-d₆) δ ppm 13.9-14.6 (m, 1H), 12.4-13.3 (m, 1H), 8.92 (t, J=5.7 Hz, 1H), 7.98 (d, J=9.1 Hz, 1H), 7.47 (d, J=9.4 Hz, 1H), 7.29-7.41 (m, 5H), 5.73 (s, 2H), 4.08 (d, J=5.6 Hz, 2H), 4.05 (s, 3H), 2.89 (s, 3H). MS (ES+) m/e 360 [M+H]⁺.

### Example 13

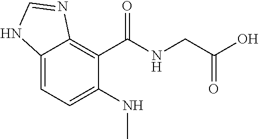

### N-{[5-(methylamino)-1H-benzimidazol-4-yl]carbonyl}glycine

### 13a) Methyl 2-amino-6-(methylamino)-3-nitrobenzoate

To a solution of methylamine (40% in water) (0.404 mL, 4.67 mmol) in tetrahydrofuran (THF) (20.0 mL) was added methyl 2-amino-6-fluoro-3-nitrobenzoate (prepared as in Example 1a) (1.00 g, 4.67 mmol). The reaction mixture was stirred overnight at ambient temperature and then concentrated in vacuo. The resulting solid was washed with ether, filtered, and dried in vacuo to afford the title compound (0.800 g, 76%) as a yellow solid. ¹H NMR (400 MHz, DMSO-d6) δ ppm 8.85 (br. s., 2H), 8.46 (br. s., 1H), 8.10 (d, J=10.1 Hz, 1H), 6.20 (d, J=9.9 Hz, 1H), 3.86 (s, 3H), 2.94 (d, J=5.1 Hz, 3H). MS (ES+) m/e 226 [M+H]⁺

### 13b) 5-(Methylamino)-1H-benzimidazole-4-carboxylic acid

To a solution of the compound from Example 13a) (0.500 g, 2.22 mmol) in methanol (100.0 mL) were added 10% palladium on charcoal (0.050 g, 0.047 mmol), hydrochloric acid (4.0 M solution in 1,4-dioxane) (1.11 mL, 4.44 mmol), and trimethyl orthoformate (0.736 mL, 6.66 mmol). The reduction was carried out under 40 psi of hydrogen gas with a Pan Shaker overnight. The reaction mixture was filtered through Celite®, and 6N aqueous sodium hydroxide (0.370 mL, 2.22 mmol) was added. The reaction mixture was stirred at ambient temperature for 0.5 h and then neutralized with 1N aqueous hydrochloric acid. The resulting precipitate was filtered, washed with water, and dried in vacuo to afford the title compound (0.240 g, 57%) as grey solid. ¹H NMR (400 MHz, DMSO-d₆) δ ppm 7.93 (br. s., 1H), 7.69 (d, J=9.09 Hz, 1H), 6.64 (d, J=8.84 Hz, 1H), 2.91 (s, 3H). MS (ES+) m/e 192 [M+H]⁺

### 13c) N-{[5-(methylamino)-1H-benzimidazol-4-yl]carbonyl}glycine

To a solution of the compound from Example 13b) (0.040 g, 0.209 mmol) in N,N-dimethylformamide (20.0 mL) were added N,N-diisopropylethylamine (0.109 mL, 0.628 mmol) and HATU (0.080 g, 0.209 mmol). Glycine ethyl ester hydrochloride (0.029 g, 0.209 mmol) was added and the mixture was stirred overnight at ambient temperature. The reaction mixture was concentrated in vacuo and the resulting residue was dissolved in methanol (20.0 mL). 6N aqueous sodium hydroxide (1.00 mL, 6.00 mmol) was added and the mixture was stirred 0.5 h at ambient temperature. Purification via preparative HPLC (YMC 75×30 mm column, 0.1% TFA in water and 0.1% TFA in acetonitrile) provided the TFA salt of the title compound (0.005 g, 6.6%) as a grey solid. ¹H NMR (400 MHz, DMSO-d₆) δ ppm 12.6 (br. s., 2H), 10.8 (br. s., 1H), 8.78 (br. s., 1H), 8.24 (s, 1H), 7.57 (d, J=8.8 Hz, 1H), 6.72 (d, J=9.1 Hz, 1H), 4.10 (d, J=5.6 Hz, 2H), 2.86 (s, 3H). MS (ES+) m/e 249[M+H]⁺.

### Example 14

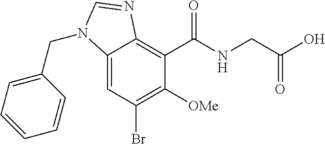

### N-{[6-bromo-5-(methyloxy)-1-(phenylmethyl)-1H-benzimidazol-4-yl]carbonyl}glycine

### 14a) Methyl 2-amino-5-bromo-6-(methyloxy)-3-nitrobenzoate

To a solution of methyl 2-amino-6-(methyloxy)-3-nitrobenzoate (prepared as in Example 1b) (0.420 g, 1.857 mmol) in dichloromethane (5.0 mL) was added bromine (0.100 mL, 1.940 mmol). After stirring 30 min. at ambient temperature, the reaction mixture was concentrated in vacuo, washed with hexanes, filtered, and dried in vacuo to afford the title compound (0.520 g, 92%) as a bright yellow solid. ¹H NMR (400 MHz, DMSO-d6) δ ppm 8.35 (s, 1H), 7.52 (br. s., 2H), 3.90 (s, 3H), 3.83 (s, 3H). MS (ES+) m/e 305/307 [M+H]⁺.

### 14b) Methyl 5-bromo-6-(methyloxy)-1H-benzimidazole-7-carboxylate

To a solution of methyl 2-amino-5-bromo-6-(methyloxy)-3-nitrobenzoate (prepared as in Example 14a) (0.500 g, 1.639 mmol) in ethyl acetate (5.0 mL) was added 10% palladium on charcoal (0.120 g, 0.113 mmol) followed by evacuation of the reaction vessel and purging with 1 atmosphere of hydrogen. Following stirring at ambient temperature for 2 h, the reaction mixture was filtered through Celite®, washed through with ethyl acetate, and concentrated in vacuo. The resulting residue was dissolved in trimethyl orthoformate (5.0 mL, 45.2 mmol) and treated with hydrochloric acid (4.0 M solution in 1,4-dioxane) (1.5 mL, 6.00 mmol). After stirring 30 min. at ambient temperature, the reaction mixture was neutralized with saturated aqueous sodium bicarbonate, diluted with brine, and extracted twice with ethyl acetate. The combined organic portions were dried over MgSO₄, filtered, concentrated in vacuo, and purified via flash column chromatography (50-100% ethyl acetate in hexanes) to afford the title compound (0.312 g, 67%) as a white solid. ¹H NMR (400 MHz, DMSO-d₆) δ ppm 12.6 (br. s., 1H), 8.31 (s, 1H), 8.15 (s, 1H), 3.94 (s, 3H), 3.83 (s, 3H). MS (ES+) m/e 285/287 [M+H]⁺.

### 14c) 6-Bromo-5-(methyloxy)-1-(phenylmethyl)-1H-benzimidazole-4-carboxylic acid

To a solution of the compound from Example 14b) (0.200 g, 0.702 mmol) in tetrahydrofuran (5.0 mL) was added benzyl bromide (0.090 mL, 0.757 mmol) followed by sodium hydride (60% dispersion in mineral oil) (0.034 g, 0.842 mmol). Following stirring at ambient temperature for 4 h, the reaction mixture was diluted with methanol (2.0 mL) and treated with 1N aqueous sodium hydroxide (2.0 mL, 2.000 mmol). After stirring overnight at ambient temperature, the reaction mixture was neutralized with 1N aqueous hydrochloric acid, diluted with brine, and extracted twice with ethyl acetate. The combined organic portions were dried over MgSO₄, filtered, concentrated in vacuo, and triturated with ethyl acetate to afford the title compound (0.180 g, 71%) as a white solid. ¹H NMR (400 MHz, DMSO-d₆) δ ppm 9.06 (br. s., 1H), 8.21 (s, 1H), 7.19-7.54 (m, 5H), 5.60 (s, 2H), 3.82 (s, 3H). MS (ES+) m/e 361/363 [M+H]⁺.

### 14d) Ethyl N-{[6-bromo-5-(methyloxy)-1-(phenylmethyl)-1H-benzimidazol-4-yl]carbonyl}glycinate

To a solution of the compound from Example 14c) (0.175 g, 0.485 mmol) and glycine ethyl ester hydrochloride (0.135 g, 0.969 mmol) in N,N-dimethylformamide (5.0 mL) were added triethylamine (0.210 mL, 1.507 mmol) and PyBOP (0.277 g, 0.533 mmol). The reaction mixture was stirred overnight at ambient temperature, quenched by water, diluted with brine, and extracted twice with EtOAc. The combined organic layers were dried over MgSO4, filtered, concentrated in vacuo, and purified via flash column chromatography (60-100% ethyl acetate in hexanes) to afford the title compound (0.201 g, 93%) as a white solid. ¹H NMR (400 MHz, DMSO-d₆) δ ppm 9.10 (t, J=5.8 Hz, 1H), 8.53 (s, 1H), 7.99 (s, 1H), 7.26-7.39 (m, 5H), 5.52 (s, 2H), 4.14 (q, J=7.1 Hz, 2H), 4.07 (d, J=5.8 Hz, 2H), 3.79 (s, 3H), 1.23 (t, J=7.1 Hz, 3H). MS (ES+) m/e 446/448 [M+H]⁺.

### 14e) N-{[6-bromo-5-(methyloxy)-1-(phenylmethyl)-1H-benzimidazol-4-yl]carbonyl}glycine

To a solution of the compound from Example 14d) (0.100 g, 0.224 mmol) in methanol (2.0 mL) and tetrahydrofuran (2.0 mL) was added 1N aqueous sodium hydroxide (1.0 mL, 1.000 mmol). After stirring 15 min. at ambient temperature, the reaction was quenched with 1N aqueous hydrochloric acid, diluted with brine, and extracted thrice with EtOAc. The combined organic layers were dried over MgSO₄, filtered, and concentrated in vacuo to afford the title compound (0.087 g, 93%) as a white solid. ¹H NMR (400 MHz, DMSO-d₆) δ ppm 9.22 (br. s., 1H), 9.05 (t, J=5.8 Hz, 1H), 8.27 (s, 1H), 7.31-7.43 (m, 5H), 5.63 (s, 2H), 4.05 (d, J=5.8 Hz, 2H), 3.85 (s, 3H). MS (ES+) m/e 418/420 [M+H]⁺.

### Example 15

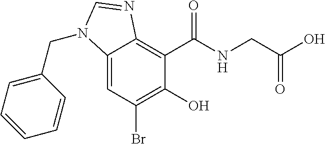

### N-{[6-bromo-5-hydroxy-1-(phenylmethyl)-1H-benzimidazol-4-yl]carbonyl}glycine

To a solution of the compound from Example 14e) (0.050 g, 0.120 mmol) in dichloromethane (5.0 mL) was added boron tribromide (1M solution in dichloromethane) (0.500 mL, 0.500 mmol). The reaction mixture was stirred overnight at ambient temperature, quenched by water, diluted with brine, and extracted thrice with ethyl acetate. The combined organic layers were dried over MgSO₄, filtered, and concentrated in vacuo to afford the title compound (0.041 g, 85%) as a white solid. ¹H NMR (400 MHz, DMSO-d₆) δ ppm 13.8 (br. s., 1H), 10.4 (t, J=5.6 Hz, 1H), 8.69 (s, 1H), 8.14 (s, 1H), 7.25-7.40 (m, 5H), 5.57 (s, 2H), 4.24 (d, J=5.6 Hz, 2H). MS (ES+) m/e 404/406 [M+H]⁺.

### Example 16

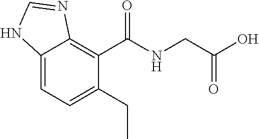

### N-[(5-ethyl-1H-benzimidazol-4-yl)carbonyl]glycine

### 16a) 1-Bromo-2-methyl-3,4-dinitrobenzene

To a suspension of 1-bromo-2-methyl-3-nitrobenzene (5.00 g, 23.1 mmol) in concentrated sulfuric acid (20.0 mL) at 0° C. was added urea nitrate (4.31 g, 34.7 mmol) portionwise in order to maintain a temperature between 0-10° C. The reaction mixture was allowed to warm slowly to ambient temperature while stirring overnight and then poured onto ice water. The resulting white precipitate was collected by filtration, washed with water, and dried in vacuo to afford the title compound (4.50 g, 75%) as a white solid. ¹H NMR (400 MHz, DMSO-d₆) δ ppm 8.19 (d, J=8.8 Hz, 1H), 8.14 (d, J=8.8 Hz, 1H), 2.38 (s, 3H). MS (ES+) m/e 261/263 [M+H]⁺.

### 16b) 6-Bromo-2,3-dinitrobenzoic acid

To a suspension of the compound from Example 16a) (1.00 g, 3.83 mmol) in concentrated sulfuric acid (5.11 mL, 96.0 mmol) was added sodium dichromate dihydrate (1.54 g, 5.17 mmol) portionwise within 1 hour. The mixture was heated to 90° C. for 2 h and quenched with ice water. The resulting white precipitate was removed by filtration. The filtrate was extracted with dichloromethane and purified via preparative HPLC (YMC 75×30 mm column, 0.1% TFA in water and 0.1% TFA in acetonitrile) to afford the title compound (0.350 g, 31%) as a white solid. ¹H NMR (400 MHz, DMSO-d₆) δ ppm 8.31 (d, J=8.8 Hz, 1H), 8.30 (d, J=8.8 Hz, 1H).

### 16c) Methyl 6-bromo-2,3-dinitrobenzoate

To a solution of the compound from Example 16b) (0.100 g, 0.344 mmol) in dichloromethane (5.0 mL) and methanol (5.0 mL) was added (trimethylsilyl)diazomethane (2.0 M solution in hexanes) (0.172 mL, 0.344 mmol) dropwise via syringe. After stirring 10 min. at ambient temperature, the reaction mixture was concentrated in vacuo, washed with diethyl ether, filtered, and dried in vacuo to afford the title compound (0.079 g, 75%) as a grey solid. ¹H NMR (400 MHz, CHLOROFORM-d) δ ppm 7.99 (s, 2H), 3.99 (s, 3H). MS (ES+) m/e 305/307 [M+H]⁺.

### 16d) Methyl 6-ethenyl-2,3-dinitrobenzoate

A solution of methyl 6-bromo-2,3-dinitrobenzoate (prepared as in Example 16c) (0.100 g, 0.328 mmol), tributyl(vinyl)tin (0.096 mL, 0.328 mmol), and tetrakis(triphenylphosphine)palladium (0.038 g, 0.033 mmol) in 1,4-dioxane (2.0 mL) was heated to 150° C. for 20 min. in a Biotage Initiator® microwave synthesizer. Upon cooling, the solution was concentrated onto silica gel and purified via flash column chromatography (0-100% ethyl acetate in hexanes) to afford the title compound (0.036 g, 44%) as an off-white solid. ¹H NMR (400 MHz, DMSO-d₆) δ ppm 8.41 (d, J=8.8 Hz, 1H), 8.30 (d, J=8.8 Hz, 1H), 6.88 (dd, J=17.2, 11.1 Hz, 1H), 6.23 (d, J=17.2 Hz, 1H), 5.77 (d, J=11.1 Hz, 1H), 3.89 (s, 3H).

### 16e) 5-Ethyl-1H-benzimidazole-4-carboxylic acid

Following the procedure of Example 13b), except substituting methyl 6-ethenyl-2,3-dinitrobenzoate (prepared as in Example 16d) for the compound from Example 13a), the TFA salt of the title compound was obtained following purification via preparative HPLC (YMC 75×30 mm column, 0.1% TFA in water and 0.1% TFA in acetonitrile) as a brown solid. ¹H NMR (400 MHz, DMSO-d₆) δ ppm 9.13 (s, 1H), 7.91 (d, J=8.6 Hz, 1H), 7.45 (d, J=8.6 Hz, 1H), 3.11 (q, J=7.5 Hz, 2H), 1.21 (t, J=7.5 Hz, 3H). MS (ES+) m/e 191 [M+H]⁺.

### 16f) N-[(5-ethyl-1H-benzimidazol-4-yl)carbonyl]glycine

Following the procedure of Example 13c), except substituting the TFA salt of 5-ethyl-1H-benzimidazole-4-carboxylic acid (prepared as in Example 16e) for the compound from Example 13b), the TFA salt of the title compound was obtained as a white solid. ¹H NMR (400 MHz, MeOD) δ ppm 9.32 (s, 1H), 7.85 (d, J=8.6 Hz, 1H), 7.60 (d, J=8.6 Hz, 1H), 4.25 (s, 2H), 3.02 (q, J=7.6 Hz, 2H), 1.33 (t, J=7.6 Hz, 3H). MS (ES+) m/e 248 [M+H]⁺.

### Example 17

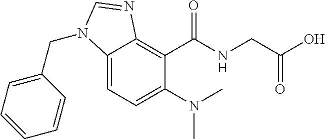

### N-{[5-(dimethylamino)-1-(phenylmethyl)-1H-benzimidazol-4-yl]carbonyl}glycine

### 17a) Methyl 2-amino-6-(dimethylamino)-3-nitrobenzoate

To a solution of methyl 2-amino-6-fluoro-3-nitrobenzoate (prepared as in Example 1a) (1.00 g, 4.67 mmol) in tetrahydrofuran (20.0 mL) was added dimethylamine (2.0 M solution in methanol) (2.40 mL, 4.80 mmol). Following stirring at ambient temperature for 4 h, the reaction mixture was concentrated in vacuo and purified via flash column chromatography (10-100% ethyl acetate in hexanes) to afford the title compound (0.962 g, 86%) as a yellow solid. ¹H NMR (400 MHz, DMSO-d₆) δ ppm 7.98 (d, J=10.0 Hz, 1H), 7.87 (br. s., 2H), 6.39 (d, J=10.0 Hz, 1H), 3.83 (s, 3H), 2.96 (s, 6H). MS (ES+) m/e 240 [M+H]⁺.

### 17b) Methyl 5-(dimethylamino)-1H-benzimidazole-4-carboxylate

To a solution of the compound from Example 17a) (0.790 g, 3.30 mmol) in ethyl acetate (10.0 mL) and methanol (5.0 mL) was added 10% palladium on charcoal (0.176 g, 0.165 mmol) followed by evacuation of the reaction vessel and purging with 1 atmosphere of hydrogen. Following stirring at ambient temperature for 2 h, the reaction mixture was filtered through Celite®, washed through with ethyl acetate, and concentrated in vacuo. The resulting residue was dissolved in methanol (5.0 mL) and treated with trimethyl orthoformate (3.65 mL, 33.0 mmol) followed by hydrochloric acid (4.0 M solution in 1,4-dioxane) (1.50 mL, 6.00 mmol). After stirring 30 min. at ambient temperature, the reaction mixture was neutralized with saturated aqueous sodium bicarbonate, diluted with brine, and extracted thrice with ethyl acetate. The combined organic portions were dried over MgSO₄, filtered, concentrated in vacuo, and purified via flash column chromatography (0-10% methanol in dichloromethane) to afford the title compound (0.667 g, 92%) as an off-white solid. ¹H NMR (400 MHz, DMSO-d₆) δ ppm 12.1 (br. s., 1H), 8.05 (s, 1H), 7.66 (d, J=8.6 Hz, 1H), 6.97 (d, J=8.6 Hz, 1H), 3.88 (s, 3H), 2.79 (s, 6H). MS (ES+) m/e 220 [M+H]⁺.

### 17c) 5-(Dimethylamino)-1-(phenylmethyl)-1H-benzimidazole-4-carboxylic acid

To a solution of the compound from Example 17b) (0.664 g, 3.03 mmol) in tetrahydrofuran (10.0 mL) was added benzyl bromide (0.390 mL, 3.28 mmol) followed by sodium hydride (60% dispersion in mineral oil) (0.145 g, 3.63 mmol). Following stirring at ambient temperature for 1 h, the reaction mixture was diluted with methanol (4.0 mL) and treated with 6N aqueous sodium hydroxide (1.5 mL, 9.00 mmol) followed by heating to reflux. After stirring overnight at reflux, the reaction mixture was neutralized with 6N aqueous hydrochloric acid, diluted with brine, and extracted twice with ethyl acetate. The aqueous portions was concentrated in vacuo, filtered, washed with dichloromethane and methanol, concentrated in vacuo, and purified via C-18 reverse phase flash column chromatography (0-100% acetonitrile in water) to afford the title compound (0.609 g, 68%) as a white solid. ¹H NMR (400 MHz, DMSO-d₆) δ ppm 9.99 (s, 1H), 8.17 (d, J=9.1 Hz, 1H), 8.00 (d, J=9.1 Hz, 1H), 7.52 (dd, J=8.1, 1.5 Hz, 2H), 7.33-7.42 (m, 3H), 5.79 (s, 2H), 3.00 (s, 6H). MS (ES+) m/e 296 [M+H]⁺.

### 17d) Ethyl N-{[5-(dimethylamino)-1-(phenylmethyl)-1H-benzimidazol-4-yl]carbonyl}glycinate

To a solution of the compound from Example 17c) (0.605 g, 2.049 mmol) and glycine ethyl ester hydrochloride (0.572 g, 4.10 mmol) in N,N-dimethylformamide (5.0 mL) were added triethylamine (0.860 mL, 6.17 mmol) and PyBOP (1.18 g, 2.268 mmol). The reaction mixture was stirred 3 d at ambient temperature, quenched by water, diluted with brine, and extracted twice with EtOAc. The combined organic layers were dried over MgSO₄, filtered, concentrated in vacuo, and purified via flash column chromatography (50-100% ethyl acetate in hexanes) to afford the title compound (0.685 g, 88%) as a white solid. ¹H NMR (400 MHz, DMSO-d₆) δ ppm 11.1 (br. s., 1H), 9.05 (br. s., 1H), 8.16 (d, J=6.5 Hz, 1H), 8.02 (s, J=6.5 Hz, 1H), 7.41 (d, J=6.8 Hz, 2H), 7.37 (t, J=7.2 Hz, 2H), 7.31 (tt, J=6.8, 1.5 Hz, 1H), 5.68 (s, 2H), 4.37 (br. s., 2H), 4.18 (q, J=7.1 Hz, 2H), 3.27 (br. s., 6H), 1.24 (t, J=7.1 Hz, 3H). MS (ES+) m/e 381 [M+H]⁺.

### 17e) N-{[5-(dimethylamino)-1-(phenylmethyl)-1H-benzimidazol-4-yl]carbonyl}glycine

To a solution of the compound from Example 17d) (0.220 g, 0.578 mmol) in methanol (2.0 mL) and tetrahydrofuran (2.0 mL) was added 1N aqueous sodium hydroxide (1.00 mL, 1.000 mmol). After stirring 15 min. at ambient temperature, the reaction mixture was neutralized with 1N aqueous hydrochloric acid and concentrated in vacuo. The resulting solid was washed with water, filtered, and dried in vacuo to afford the title compound (0.178 g, 87%) as a white solid. ¹H NMR (400 MHz, DMSO-d₆) δ ppm 14.0 (br. s., 1H), 11.0 (br. s., 1H), 9.06 (s, 1H), 8.14 (d, J=8.6 Hz, 1H), 7.97 (br. s., 1H), 7.40 (d, J=7.1 Hz, 2H), 7.36 (t, J=7.6 Hz, 2H), 7.31 (t, J=7.1 Hz, 1H), 5.68 (s, 2H), 4.29 (d, J=5.1 Hz, 2H), 3.24 (br. s., 6H). MS (ES+) m/e 353 [M+H]⁺.

### Example 18

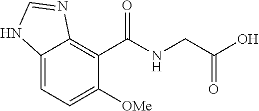

### N-{[5-(methyloxy)-1H-benzimidazol-4-yl]carbonyl}glycine

### 18a) Methyl 5-(methyloxy)-1H-benzimidazole-4-carboxylate

To a solution of methyl 2-amino-6-(methyloxy)-3-nitrobenzoate (prepared as in Example 1b) (0.452 g, 1.998 mmol) in ethyl acetate (10.0 mL) was added 10% palladium on charcoal (0.106 g, 0.100 mmol) followed by evacuation of the reaction vessel and purging with 1 atmosphere of hydrogen. Following stirring at ambient temperature for 4 h, the reaction mixture was filtered through Celite®, washed through with ethyl acetate, and concentrated in vacuo. The resulting residue was dissolved in methanol (5.0 mL) and treated with trimethyl orthoformate (2.20 mL, 19.90 mmol) followed by hydrochloric acid (4.0 M solution in 1,4-dioxane) (1.00 mL, 4.00 mmol). After stirring 30 min. at ambient temperature, the reaction mixture was neutralized with saturated aqueous sodium bicarbonate, diluted with brine, and extracted thrice with ethyl acetate. The combined organic portions were dried over MgSO₄, filtered, concentrated in vacuo, and triturated with 1:1:1 methanol/diethyl ether/hexanes to afford the title compound (0.347 g, 84%) as a pale orange solid. ¹H NMR (400 MHz, DMSO-d₆) δ ppm 12.2 (br. s., 1H), 8.13 (s, 1H), 7.84 (d, J=9.1 Hz, 1H), 7.05 (d, J=9.1 Hz, 1H), 3.87 (s, 3H), 3.87 (s, 3H). MS (ES+) m/e 207 [M+H]⁺.

### 18b) 5-(Methyloxy)-1H-benzimidazole-4-carboxylic acid

To a solution of the compound from Example 18a) (0.342 g, 1.659 mmol) in methanol (2.0 mL) and tetrahydrofuran (2.0 mL) was added 1N aqueous sodium hydroxide (2.0 mL, 2.000 mmol). After stirring overnight at ambient temperature, the reaction was quenched with 1N aqueous hydrochloric acid, concentrated in vacuo, diluted with methanol, filtered, and concentrated in vacuo to afford the title compound (0.304 g, 95%) as an off-white solid. ¹H NMR (400 MHz, DMSO-d₆) δ ppm 13.9 (br. s., 1H), 9.31 (s, 1H), 7.98 (d, J=9.1 Hz, 1H), 7.41 (d, J=9.1 Hz, 1H), 3.94 (s, 3H). MS (ES+) m/e 193 [M+H]⁺.

### 18c) Ethyl N-{[5-(methyloxy)-1H-benzimidazol-4-yl]carbonyl}glycinate

To a solution of the compound from Example 18b) (0.300 g, 1.561 mmol) and glycine ethyl ester hydrochloride (0.436 g, 3.12 mmol) in N,N-dimethylformamide (5.0 mL) were added triethylamine (0.660 mL, 4.74 mmol) and PyBOP (0.900 g, 1.729 mmol). The reaction mixture was stirred overnight at ambient temperature, quenched by water, filtered, washed with EtOAc, and dried in vacuo to afford the title compound (0.336 g, 78%) as an off-white solid. ¹H NMR (400 MHz, DMSO-d₆) δ ppm 12.3 (s, 1H), 8.82 (t, J=5.6 Hz, 1H), 8.03 (s, 1H), 7.81 (d, J=8.8 Hz, 1H), 7.11 (d, J=8.8 Hz, 1H), 4.15 (t, J=7.1 Hz, 2H), 4.12 (d, J=5.6 Hz, 2H), 4.01 (s, 3H), 1.22 (t, J=7.1 Hz, 3H). MS (ES+) m/e 278 [M+H]⁺.

### 18d) N-{[5-(methyloxy)-1H-benzimidazol-4-yl]carbonyl}glycine

To a solution of the compound from Example 18c) (0.180 g, 0.649 mmol) in methanol (2.0 mL) and tetrahydrofuran (2.0 mL) was added 1N aqueous sodium hydroxide (1.5 mL, 1.500 mmol). After stirring 30 min. at ambient temperature, the reaction was quenched with 1N aqueous hydrochloric acid, filtered, washed with water and methanol, and dried in vacuo to afford the title compound (0.155 g, 96%) as an off-white solid. ¹H NMR (400 MHz, DMSO-d₆) δ ppm 12.7 (br. s., 1H), 12.3 (br. s., 1H), 8.77 (t, J=5.6 Hz, 1H), 8.04 (s, 1H), 7.80 (d, J=9.1 Hz, 1H), 7.10 (d, J=9.1 Hz, 1H), 4.06 (d, J=5.6 Hz, 2H), 4.00 (s, 3H). MS (ES+) m/e 250 [M+H]⁺.

### Example 19

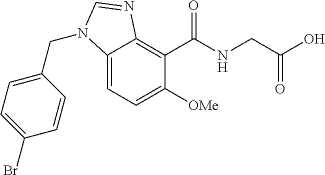

### N-{[1-[(4-bromophenyl)methyl]-5-(methyloxy)-1H-benzimidazol-4-yl]carbonyl}glycine

### 19a) Methyl 1-[4-bromophenyl)methyl]-5-(methyloxy)-1H-benzimidazole-4-carboxylate

To a suspension of methyl 5-(methyloxy)-1H-benzimidazole-4-carboxylate (prepared as in Example 1c) (0.920 g, 4.46 mmol) in N,N-dimethylformamide (20.0 mL) at 0° C. was added sodium hydride (60% dispersion in mineral oil) (0.357 g, 8.92 mmol). After the mixture was stirred at 0° C. for 10 min., 4-bromobenzyl bromide (1.67 g, 6.69 mmol) was added. Upon removal of the cooling bath, the reaction was stirred at ambient temperature for 3 h, quenched with ice water, and extracted with ethyl acetate. The organic extract was dried over MgSO₄, filtered, concentrated in vacuo, and purified via flash column chromatography (0-100% ethyl acetate in hexanes) to afford the title compound (0.650 g, 39%) as a colorless oil. ¹H NMR (400 MHz, CHLOROFORM-d) δ ppm 8.02 (s, 1H), 7.48 (d, J=8.6 Hz, 2H), 7.22 (d, J=8.8 Hz, 1H), 7.03 (d, J=8.6 Hz, 2H), 6.97 (d, J=8.8 Hz, 1H), 5.31 (s, 2H), 4.06 (s, 3H), 3.91 (s, 3H). MS (ES+) m/e 375/377 [M+H]⁺.

### 19b) 1-[(4-Bromophenyl)methyl]-5-(methyloxy)-1H-benzimidazole-4-carboxylic acid

Following the procedure of Example 11d), except substituting the compound from Example 19a) for the compound from Example 11c), the title compound was obtained as a white solid. ¹H NMR (400 MHz, DMSO-d6) δ ppm 8.45 (s, 1H), 7.54 (dd, J=8.7, 2.7 Hz, 3H), 7.26 (d, J=8.6 Hz, 2H), 7.07 (d, J=9.1 Hz, 1H), 5.49 (s, 2H), 3.80 (s, 3H). MS (ES+) m/e 361/363 [M+H]⁺.

### 19c) N-{[1-[(4-bromophenyl)methyl]-5-(methyloxy)-1H-benzimidazol-4-yl]carbonyl}glycine

Following the procedure of Example 13c), except substituting the compound from Example 19b) for the compound from Example 13b), the TFA salt of the title compound was obtained as a white solid. ¹H NMR (400 MHz, MeOD) δ ppm 9.47 (s, 1H), 7.89 (d, J=9.3 Hz, 1H), 7.56 (d, J=8.6 Hz, 2H), 7.51 (d, J=9.3 Hz, 1H), 7.34 (d, J=8.6 Hz, 2H), 5.70 (s, 2H), 4.20 (s, 2H), 4.11 (s, 3H). MS (ES+) m/e 418/420 [M+H]⁺.

### Example 20

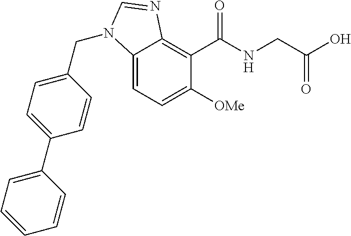

### N-{[1-(4-biphenylylmethyl)-5-(methyloxy)-1H-benzimidazol-4-yl]carbonyl}glycine

A suspension of the compound from Example 19c) (0.033 g, 0.079 mmol), phenylboronic acid (0.0106 g, 0.087 mmol), potassium carbonate (0.0327 g, 0.237 mmol), and tetrakis(triphenylphosphine)palladium(O) (0.0091 g, 0.0079 mmol) in 1,4-dioxane (3.0 mL) and water (1.0 mL) was heated to 100° C. for 20 min in a Biotage Initiator® microwave synthesizer. Upon cooling, the reaction mixture was purified via preparative HPLC (YMC 75×30 mm column, 0.1% TFA in water and 0.1% TFA in acetonitrile) to afford the TFA salt of the title compound (0.007 g, 21%) as a white solid. ¹H NMR (400 MHz, MeOD) δ ppm 9.52 (s, 1H), 8.90 (t, J=5.6 Hz, 1H), 7.97 (d, J=9.1 Hz, 1H), 7.67 (d, J=8.3 Hz, 2H), 7.58 (d, J=7.1 Hz, 1H), 7.47-7.55 (m, 3H), 7.42 (t, J=7.6 Hz, 2H), 7.27-7.37 (m, 1H), 5.78 (s, 2H), 4.22 (s, 2H), 4.12 (s, 3H). MS (ES+) m/e 416 [M+H]⁺.

### Example 21

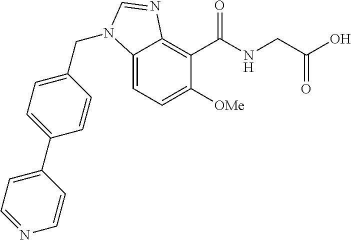

### N-[5-(methyloxy)-1-{[4-(4-pyridinyl)phenyl]methyl}-1H-benzimidazol-4-yl)carbonyl]glycine

Following the procedure of Example 20, except substituting 4-pyridinylboronic acid for phenylboronic acid, the bis-TFA salt of the title compound was obtained as a white solid. ¹H NMR (400 MHz, MeOD) δ ppm 9.56 (s, 1H), 8.81 (d, J=6.8 Hz, 2H), 8.30 (d, J=6.8 Hz, 2H), 7.97 (d, J=8.6 Hz, 2H), 7.90 (d, J=9.3 Hz, 1H), 7.62 (d, J=8.6 Hz, 2H), 7.48 (d, J=9.3 Hz, 1H), 5.85 (s, 2H), 4.17 (s, 2H), 4.08 (s, 3H). MS (ES+) m/e 417 [M+H]⁺.

### Example 22

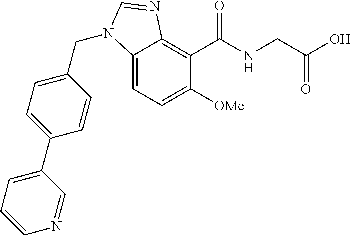

### N-[(5-(methyloxy)-1-{[4-(3-pyridinyl)phenyl]methyl}-1H-benzimidazol-4-yl)carbonyl]glycine

Following the procedure of Example 20, except substituting 3-pyridinylboronic acid for phenylboronic acid, the bis-TFA salt of the title compound was obtained as a white solid. ¹H NMR (400 MHz, MeOD) δ ppm 9.51 (s, 1H), 9.06 (d, J=2.0 Hz, 1H), 8.67-8.80 (m, 2H), 8.02 (dd, J=8.2, 5.7 Hz, 1H), 7.86 (d, J=9.3 Hz, 1H), 7.78 (d, J=8.6 Hz, 2H), 7.56 (d, J=8.3 Hz, 2H), 7.43 (d, J=9.3 Hz, 1H), 5.78 (s, 2H), 4.12 (s, 2H), 4.03 (s, 3H). MS (ES+) m/e 417 [M+H]⁺.

### Example 23

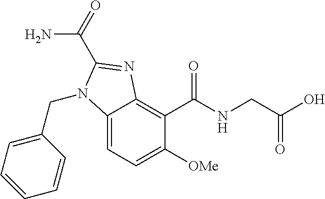

### N-{[2-(aminocarbonyl)-5-(methyloxy)-1-(phenylmethyl)-1H-benzimidazol-4-yl]carbonyl}glycine

### 23a) Methyl 5-(methyloxy)-2-(trichloromethyl)-1H-benzimidazole-4-carboxylate

To a suspension of methyl 2-amino-6-(methyloxy)-3-nitrobenzoate (prepared as in Example 1b) (1.00 g, 4.42 mmol) in acetic acid (50.0 mL) was added 10% palladium on charcoal (0.094 g, 0.088 mmol). The reduction was carried out under 40 psi of hydrogen gas with a Parr Shaker overnight. The reaction mixture was filtered through Celite® and methyl trichloroacetimidate (0.780 g, 4.42 mmol) was added. The reaction was stirred at ambient temperature for 1 h, quenched with ice water, and extracted with ethyl acetate. The extract was dried over MgSO₄, filtered, concentrated in vacuo, and purified via flash column chromatography (0-100% ethyl acetate in hexanes) to afford the title compound (0.210 g, 15%) as a yellow solid. ¹H NMR (400 MHz, CHLOROFORM-d) δ ppm 11.0 (br. s., 1H), 8.00 (d, J=9.1 Hz, 1H), 7.07 (d, J=9.1 Hz, 1H), 4.03 (s, 3H), 4.02 (s, 3H). MS (ES+) m/e 323 [M+H]⁺.

### 23b) Methyl 2-cyano-5-(methyloxy)-1H-benzimidazole-4-carboxylate

To condensed ammonia (10.0 mL, 462 mmol) at −78° C. was added methyl 5-(methyloxy)-2-(trichloromethyl)-1H-benzimidazole-4-carboxylate (prepared as in Example 23a) (0.600 g, 1.85 mmol). Following stirring at −78° C. for 5 min., the reaction mixture was allowed to warm slowly to ambient temperature. Upon evaporation of the ammonia, the resulting solid was washed the dichloromethane, filtered, and dried in vacuo to afford the title compound (0.250 g, 58%) as a white solid. ¹H NMR (400 MHz, DMSO-d₆) δ ppm 7.97 (d, J=9.3 Hz, 1H), 7.29 (d, J=9.3 Hz, 1H), 3.93 (s, 3H), 3.89 (s, 3H). MS (ES+) m/e 232 [M+H]⁺.

### 23c) Methyl 2-cyano-5-(methyloxy)-1-(phenylmethyl)-1H-benzimidazole-4-carboxylate

To a solution of the compound from Example 23b) (0.100 g, 0.433 mmol) in N,N-dimethylformamide (10.0 mL) at 0° C. was added sodium hydride (60% dispersion in mineral oil) (0.035 g, 0.865 mmol). After the mixture was stirred at 0° C. for 5 min., benzyl bromide (0.057 mL, 0.476 mmol) was added. Upon removal of the cooling bath, the reaction was stirred overnight at ambient temperature, quenched with ice water, and extracted with ethyl acetate. The organic extract was dried over MgSO₄, filtered, concentrated in vacuo, and purified via flash column chromatography (0-100% ethyl acetate in hexanes) to afford the title compound (0.042 g, 30%) as a white solid. ¹H NMR (400 MHz, MeOD) δ ppm 7.69 (d, J=9.1 Hz, 1H), 7.31-7.39 (m, 4H), 7.24-7.29 (m, 2H), 5.65 (s, 2H), 3.94 (s, 3H), 3.89 (s, 3H). MS (ES+) m/e 322 [M+H]⁺.

### 23d) 2-(Aminocarbonyl)-5-(methyloxy)-1-(phenylmethyl)-1H-benzimidazole-4-carboxylic acid

To a solution of methyl 2-cyano-5-(methyloxy)-1-(phenylmethyl)-1H-benzimidazole-4-carboxylate (prepared as in Example 23c) (0.150 g, 0.467 mmol) in methanol (10.0 mL) was added 6N aqueous sodium hydroxide (0.389 mL, 2.335 mmol). The mixture was heated to 50° C. for 3 h, allowed to cool to ambient temperature, and purified via preparative HPLC (YMC 75×30 mm column, 0.1% TFA in water and 0.1% TFA in acetonitrile) to afford the TFA salt of the title compound (0.066 g, 43%) as a yellow solid. ¹H NMR (400 MHz, DMSO-d₆) δ ppm 13.0 (br. s., 1H), 8.20 (s, 1H), 7.95 (s, 1H), 7.68 (d, J=9.1 Hz, 1H), 7.28-7.35 (m, 2H), 7.17-7.27 (m, 4H), 5.97 (s, 2H), 3.83 (s, 3H). MS (ES+) m/e 326 [M+H]⁺.

### 23e) N-{[2-(aminocarbonyl)-5-(methyloxy)-1-(phenylmethyl)-1H-benzimidazol-4-yl]carbonyl}glycine

Following the procedure of Example 13c), except substituting the compound from Example 23d) for the compound from Example 13b), the TFA salt of the title compound was obtained as a white solid. ¹H NMR (400 MHz, MeOD) δ ppm 7.72 (d, J=9.3 Hz, 1H), 7.32 (d, J=9.3 Hz, 1H), 7.19-7.29 (m, 5H), 6.00 (s, 2H), 4.22 (s, 2H), 3.97 (s, 3H). MS (ES+) m/e 383 [M+H]⁺.

### Biological Background:

The following references set out information about the target enzymes, HIF prolyl hydroxylases, and methods and materials for measuring inhibition of same by small molecules.
M. Hirsilä, P. Koivunen, V. Günzler, K. I. Kivirikko, and J. Myllyharju “Characterization of the Human Prolyl 4-Hydroxylases That Modify the Hypoxia-inducible Factor” J. Biol. Chem., 2003, 278, 30772-30780.
C. Willam, L. G. Nicholls, P. J. Ratcliffe, C. W. Pugh, P. H. Maxwell “The prolyl hydroxylase enzymes that act as oxygen sensors regulating destruction of hypoxia-inducible factor α” Advan. Enzyme Regal., 2004, 44, 75-92
M. S. Wiesener, J. S. Jürgensen, C. Rosenberger, C. K. Scholze, J. H. Hörstrup, C. Warnecke, S. Mandriota, I. Bechmann, U. A. Frei, C. W. Pugh, P. J. Ratcliffe, S. Bachmann, P. H. Maxwell, and K.-U. Eckardt “Widespread hypoxia-inducible expression of HIF-2α in distinct cell populations of different organs” FASEB J., 2003, 17, 271-273.
S. J. Klaus, C. J. Molineaux, T. B. Neff, V. Guenzler-Pukall, I. Lansetmo Parobok, T. W. Seeley, R. C. Stephenson “Use of hypoxia-inducible factor α (HIFα) stabilizers for enhancing erythropoiesis” PCT Int. Appl. (2004), WO 2004108121 A1
C. Warnecke, Z. Zaborowska, J. Kurreck, V. A. Erdmann, U. Frei, M. Wiesener, and K.-U. Eckardt “Differentiating the functional role of hypoxia-inducible factor (HIF)-1α and HIF-2α (EPAS-1) by the use of RNA interference: erythropoietin is a HIF-2α target gene in Hep3B and Kelly cells” FASEB J., 2004, 18, 1462-1464.
   For the expression of EGLN3 see:
R. K. Bruick and S. L. McKnight “A Conserved Family of Prolyl-4-Hydroxylases That Modify HIF” Science, 2001, 294, 1337-1340.
   For the expression of HIF2α-CODD see:
a) P. Jaakkola, D. R. Mole, Y.-M. Tian, M. I. Wilson, J. Gielbert, S. J. Gaskell, A. von Kriegsheim, H. F. Hebestreit, M. Mukherji, C. J. Schofield, P. H. Maxwell, C. W. Pugh, P, J. Ratcliffe “Targeting of HIF-a to the von Hippel-Lindau Ubiquitylation Complex by O₂-Regulated Prolyl Hydroxylation” Science, 2001, 292, 468-472.
b) M. Ivan, K. Kondo, H. Yang, W. Kim, J. Valiando, M. Ohh, A. Salic, J. M. Asara, W. S. Lane, W. G. Kaelin Jr. “HIFα Targeted for VHL-Mediated Destruction by Proline Hydroxylation: Implications for O₂Sensing” Science, 2001, 292, 464-468.
   For the expression of VHL, elongin b and elongin c see:
A. Pause, S. Lee, R. A. Worrell, D. Y. T. Chen, W. H. Burgess, W. M. Linehan, R. D. Klausner “The von Hippel-Lindau tumor-suppressor gene product forms a stable complex with human CUL-2, a member of the Cdc53 family of proteins” Proc. Natl. Acad. Sci. USA, 1997, 94, 2156-2161.

### Biological Assay(s)

### EGLN3 Assay

### Materials:

His-MBP-EGLN3 (6HisMBPAttB1EGLN3(1-239)) was expressed in E. Coli and purified from an amylase affinity column. Biotin-VBC [6HisSumoCysVHL(2-213), 6HisSumoElonginB(1-118), and 6HisSumoElonginC(1-112)] and His-GB1-HIF2α-CODD (6HisGB1tevHIF2A(467-572)) were expressed from E. Coli.

### Method:

Cy5-labelled HIF2a CODD, and a biotin-labeled VBC complex were used to determine EGLN3 inhibition. EGLN3 hydroxylation of the Cy5CODD substrate results in its recognition by the biotin-VBC. Addition of a Europium/streptavidin (Eu/SA) chelate results in proximity of Eu to Cy5 in the product, allowing for detection by energy transfer. A ratio of Cy5 to Eu emission (LANCE Ratio) is the ultimate readout, as this normalized parameter has significantly less variance than the Cy5 emission alone.

Then 50 mL of inhibitors in DMSO (or DMSO controls) were stamped into a 384-well low volume Corning NBS plate, followed by addition of 2.5 μL of enzyme [50 mL buffer (50 mM HEPES/50 mM KCl)+1 mL of a 10 mg/mL BSA in buffer+6.25 μL of a 10 mg/mL FeCl₂ solution in water+100 μL of a 200 mM solution of ascorbic acid in water+15.63 ®L EGLN3] or control [50 mL buffer+1 mL of a 10 mg/mL BSA in buffer+6.25 μL of a 10 mg/mL FeCl₂ solution in water+100 μL of a 200 mM solution of ascorbic acid in water]. Following a 3 minutes incubation, 2.5 μL of substrate [50 mL Buffer+68.6 μL biotin-VBC+70.4 μL Eu (at 710 μg/mL stock)+91.6 μL Cy5CODD+50 μL of a 20 mM solution of 2-oxoglutaric acid in water+0.3 mM CHAPS] was added and incubated for 30 minutes. The plate was loaded into a PerkinElmer Viewlux for imaging. For dose response experiments, normalized data were fit by ABASE/XC50 using the equation y=a+(b−a)/(1+(10̂x/10̂c)̂d), where a is the minimum % activity, b is the maximum % activity, c is the pIC₅₀, and d is the Hill slope.

The IC₅₀ for exemplified compounds in the EGLN3 assay ranged from approximately 1-100 nanomolar. This range represents the data accumulated as of the time of the filing of this initial application. Later testing may show variations in IC₅₀ data due to variations in reagents, conditions and variations in the method(s) used from those given herein above. So this range is to be viewed as illustrative, and not a absolute set of numbers.

Measure Epo protein produced by Hep3B cell line using ELISA method.

Hep3B cells obtained from the American Type Culture Collection (ATCC) are seeded at 2×10̂4 cells/well in Dulbecco's Modified Eagle Medium (DMEM)+10% FBS in 96-well plates. Cells are incubated at 37degC/5% CO2/90% humidity (standard cell culture incubation conditions). After overnight adherence, medium is removed and replaced with DMEM without serum containing test compound or DMSO negative control. Following 48 hours incubation, cell culture medium is collected and assayed by ELISA to quantitate Epo protein.

The EC₅₀ for exemplar compounds in the Hep3B ELISA assay ranged from approximately 1-20 micromolar using the reagents and under the conditions outlined herein above. This range represents the data accumulated as of the time of the filing of this initial application. Later testing may show variations in EC₅₀ data due to variations in reagents, conditions and variations in the method(s) used from those given herein above. So this range is to be viewed as illustrative, and not a absolute set of numbers.

These compound are believed to be useful in therapy as defined above and to not have unacceptable or untoward effects when used in compliance with a permitted therapeutic regime.

The foregoing examples and assay have been set forth to illustrate the invention, not limit it. What is reserved to the inventors is to be determined by reference to the claims.

1. A compound of formula (I): 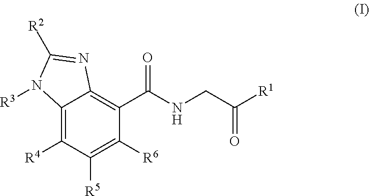
wherein:
R¹ is —NR⁷R⁸ or —OR⁹;
R² is selected from the group consisting of hydrogen, nitro, cyano, —C(O)R¹², —C(O)OR¹²—OR¹², —SR¹², —S(O)R¹²—NR¹⁰R¹¹, —CONR¹⁰R¹¹, —N(R¹⁰)C(O)R¹², —N(R¹⁰)C(O)OR¹², —OC(O)NR¹⁰R¹¹, —N(R¹⁰)C(O)N¹⁰R¹¹, —SO₂NR¹⁰R¹¹, —N(R¹⁰)SO₂R¹², C₁-C₁₀ alkyl, C₂-C₁₀ alkenyl, C₂-C₁₀ alkynyl, C₃-C₈ cycloalkyl, C₃-C₈ heterocycloalkyl, C₅-C₈ cycloalkenyl, aryl, and heteroaryl;
R³ is selected from the group consisting of hydrogen, —C(O)R¹², —C(O)OR¹², —S(O)₂R¹², —CONR¹⁰R¹¹, —SO₂NR¹⁰R¹¹, C₁-C₁₀ alkyl, C₂-C₁₀ alkenyl, C₂-C₁₀ alkynyl, C₃-C₈ cycloalkyl, C₃-C₈ heterocycloalkyl, C₅-C₈ cycloalkenyl, aryl, and heteroaryl;
R⁴, R⁵, and R⁶ are each independently selected from the group consisting of hydrogen, nitro, cyano, halogen, —C(O)R¹², —C(O)OR¹², —OR¹², —SR¹², —S(O)R¹², —S(O)₂R¹², —NR¹⁰R¹¹, —CONR¹⁰R¹¹, —N(R¹⁰)C(O)R¹², —N(R¹⁰)C(O)OR¹², —OC(O)NR¹⁰R¹¹, —N(R¹⁰)C(O)N¹⁰R¹¹, —P(O)(OR¹²)₂, —SO₂NR¹⁰R¹¹, —N(R¹⁰)SO₂R¹², C₁-C₁₀ alkyl, C₂-C₁₀ alkenyl, C₂-C₁₀ alkynyl, C₃-C₈ cycloalkyl, C₃-C₈ heterocycloalkyl, C₅-C₈ cycloalkenyl, aryl, and heteroaryl;
R⁷ and R⁸ are each independently selected from the group consisting of hydrogen, C₁-C₆ alkyl, C₂-C₆ alkenyl, C₂-C₆ alkynyl, C₃-C₆ cycloalkyl, C₃-C₆ heterocycloalkyl, aryl, and heteroaryl;
R⁹ is hydrogen, or a cation, or C₁-C₄ alkyl;
R¹⁰ and R¹¹ are each independently selected from the group consisting of hydrogen, C₁-C₁₀ alkyl, C₃-C₈ cycloalkyl, C₁-C₁₀ alkyl-C₃-C₈ cycloalkyl, C₃-C₈heterocycloalkyl, alkyl-C₃-C₈ heterocycloalkyl, aryl, C₁-C₁₀ alkyl-aryl, heteroaryl, C₁-C₁₀ alkyl-heteroaryl, —CO(C₁-C₄ alkyl), —CO(C₃-C₆ cycloalkyl), —CO(C₃-C₆ heterocycloalkyl), —CO(aryl), —CO(heteroaryl), and —SO₂(C₁-C₄ alkyl); or R¹⁰ and R¹¹ taken together with the nitrogen to which they are attached form a 5- or 6- or 7-membered saturated ring optionally containing one other heteroatom which is oxygen, nitrogen or sulfur;
each R¹² is independently selected from the group consisting of hydrogen, C₁-C₁₀ alkyl, C₂-C₁₀ alkenyl, C₂-C₁₀ alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈ heterocycloalkyl, aryl, C₁-C₁₀ alkyl-aryl, heteroaryl, and C₁-C₁₀ alkyl-heteroaryl;
any carbon or heteroatom of R², R³, R⁴, R⁵, R⁶, R⁷, R⁸, R⁹, R¹⁰, R¹¹, or R¹² is unsubstituted or, where possible, is substituted with one or more substituents independently selected from C₁-C₆ alkyl, aryl, heteroaryl, halogen, —OR¹², —NR¹⁰R¹¹, cyano, nitro, —C(O)R¹², —C(O)OR¹², —SR¹², —S(O)R¹², —S(O)R¹², —CONR¹⁰R¹¹, —N(R¹⁰)C(O)OR¹², —OC(O)NR¹⁰R¹¹, —N(R¹⁰)C(O)NR¹⁰R¹¹, —SO₂NR¹⁰R¹¹, N(R¹⁰)SO₂R¹², C₂-C₁₀ alkenyl, C₂-C₁₀ alkynyl, C₃-C₈ cycloalkyl, C₃-C₈ heterocycloalkyl, C₅-C₈ cycloalkenyl, aryl or heteroaryl,
wherein, R¹⁰, R¹¹, and R¹² are the same as defined above;
or a pharmaceutically acceptable salt or solvate thereof.

2. A compound according to claim 1 wherein:
R¹ is —OR⁹;
R² is selected from the group consisting of hydrogen, cyano, —C(O)R¹², —C(O)OR¹², —CONR¹⁰R¹¹, C₁-C₆ alkyl, C₂-C₆ alkenyl, C₂-C₆ alkynyl, C₃-C₆ cycloalkyl, C₃-C₆ heterocycloalkyl, aryl, and heteroaryl;
R³ is selected from the group consisting of hydrogen, —S(O)₂R¹², C₁-C₁₀ alkyl, C₂-C₁₀ alkenyl, C₂-C₁₀ alkynyl, C₃-C₈ cycloalkyl, C₃-C₈ heterocycloalkyl, C₅-C₈ cycloalkenyl, aryl, and heteroaryl;
R⁴, R⁵, and R⁶ are each independently selected from the group consisting of hydrogen, cyano, halogen, —OR¹², —NR¹⁰R¹¹, —CONR¹⁰R¹¹, C₁-C₆ alkyl, C₃-C₆ cycloalkyl, C₃-C₆ heterocycloalkyl, aryl, and heteroaryl;
R⁹ is hydrogen, or a cation;
R¹⁰ and R¹¹ are each independently selected from the group consisting of hydrogen, C₁-C₆ alkyl, C₃-C₆ cycloalkyl, C₃-C₆ heterocycloalkyl, aryl, heteroaryl, —CO(C₁-C₄ alkyl), —CO(C₃-C₆ cycloalkyl), —CO(C₃-C₆ heterocycloalkyl), —CO(aryl), —CO(heteroaryl), and —SO₂(C₁-C₄ alkyl); or R¹⁰ and R¹¹ taken together with the nitrogen to which they are attached form a 5- or 6- or 7-membered saturated ring optionally containing one other heteroatom which is oxygen, nitrogen or sulfur;
each R¹² is independently selected from the group consisting of hydrogen, C₁-C₆ alkyl, C₂-C₆ alkenyl, C₂-C₆ alkynyl, —CO(C₁-C₄ alkyl), —CO(aryl), —CO(heteroaryl), —CO(C₃-C₆ cycloalkyl), —CO(C₃-C₆ heterocycloalkyl), C₃-C₆ cycloalkyl, C₃-C₆ heterocycloalkyl, aryl, and heteroaryl;
any carbon or heteroatom of R², R³, R⁴, R⁵, R⁶, R⁹, R¹⁰, R¹¹, or R¹² is unsubstituted or, where possible, is substituted with one or more substituents independently selected from C₁-C₆ alkyl, aryl, heteroaryl, halogen, —OR¹², —NR¹⁰R¹¹, cyano, —C(O)R¹², —C(O)OR¹², —CONR¹⁰R¹¹, —N(R¹⁰)SO₂R¹², —N(R¹⁰)C(O)OR¹², —OC(O)NR¹⁰R¹¹, —N(R¹⁰)C(O)NR¹⁰R¹¹, —SO₂NR¹⁰R¹¹, —N(R¹⁰)SO₂R¹², C₂-C₆ alkenyl, C₂-C₆ alkynyl, C₃-C₆ cycloalkyl, C₃-C₆ heterocycloalkyl, C₅-C₈ cycloalkenyl, aryl, or heteroaryl, wherein R¹⁰, R¹¹, and R¹² are the same as defined above;
or a pharmaceutically acceptable salt or solvate thereof.

3. A compound according to claim 1 wherein:
R¹ is —OR⁹;
R² is selected from the group consisting of hydrogen, cyano, —CONR¹⁰R¹¹, C₁-C₆ alkyl, C₃-C₆ cycloalkyl, C₃-C₆ heterocycloalkyl, aryl, and heteroaryl;
R³ is selected from the group consisting of hydrogen, C₁-C₆ alkyl, C₃-C₆ cycloalkyl, C₃-C₆ heterocycloalkyl, C₅-C₆ cycloalkenyl, aryl, and heteroaryl;
R⁴ is hydrogen;
R⁵ and R⁶ are each independently selected from the group consisting of hydrogen, cyano, halogen, —OR¹², —NR¹⁰R¹¹, —CONR¹⁰R¹¹, C₁-C₆ alkyl, C₃-C₆ cycloalkyl, C₃-C₆ heterocycloalkyl, aryl, and heteroaryl;
R⁹ is hydrogen, or a cation;
R¹⁰ and R¹¹ are each independently selected from the group consisting of hydrogen, C₁-C₆ alkyl, C₃-C₆ cycloalkyl, C₃-C₆ heterocycloalkyl, aryl, and heteroaryl; or R¹⁰ and R¹¹ taken together with the nitrogen to which they are attached form a 5- or 6- or 7-membered saturated ring optionally containing one other heteroatom which is oxygen, nitrogen or sulfur;
each R¹² is independently selected from the group consisting of hydrogen, C₁-C₆ alkyl, C₃-C₆ cycloalkyl, C₃-C₆ heterocycloalkyl, aryl, and heteroaryl;
any carbon or heteroatom of R², R³, R⁵, R⁶, R¹⁰, R¹¹ or R¹² is unsubstituted or, where possible, is substituted with one or more substituents independently selected from C₁-C₆ alkyl, aryl, heteroaryl, halogen, —OR¹², —NR¹⁰R¹¹, cyano, —C(O)R¹², —C(O)OR¹², —CONR¹⁰R¹¹, —N(R¹⁰)C(O)R¹², N(R¹⁰)C(O)OR¹², —OC(O)NR¹⁰R¹¹, N(R¹⁰)C(O)NR¹⁰R¹¹, SO₂NR¹⁰R¹¹, N(R¹⁰)SO₂R¹², C₂-C₆ alkenyl, C₂-C₆ alkynyl, C₃-C₆ cycloalkyl, C₃-C₆ heterocycloalkyl, C₅-C₈ cycloalkenyl, aryl, or heteroaryl, wherein R¹⁰, R¹¹ and R¹² are the same as defined above;
or a pharmaceutically acceptable salt or solvate thereof.

4. A compound according to claim 1 which is:
N-{[5-hydroxy-1-(phenylmethyl)-1H-benzimidazol-4-yl]carbonyl}glycine;
N-{[5-(methyloxy)-1-(phenylmethyl)-1H-benzimidazol-4-yl]carbonyl}glycine;
N-({5-[(phenylmethyl)oxy]-1H-benzimidazol-4-yl}carbonyl)glycine;
N-[(5-hydroxy-1H-benzimidazol-4-yl)carbonyl]glycine;
N-{[1-[(2-chlorophenyl)methyl]-5-(methyloxy)-1H-benzimidazol-4-yl]carbonyl}glycine;
N-({1-[(2-chlorophenyl)methyl]-5-hydroxy-1H-benzimidazol-4-yl}carbonyl)glycine;
N-{[5-fluoro-1-(phenylmethyl)-1H-benzimidazol-4-yl]carbonyl}glycine;
N-{[(5-fluoro-1H-benzimidazol-4-yl]carbonyl}glycine;
N-{[1-(phenylmethyl)-1H-benzimidazol-4-yl]carbonyl}glycine;
N-(1H-benzimidazol-4-ylcarbonyl)glycine;
N-{[5-(methyloxy)-2-phenyl-1-(phenylmethyl)-1H-benzimidazol-4-yl]carbonyl}glycine;
N-{[2-methyl-5-(methyloxy)-1-(phenylmethyl)-1H-benzimidazol-4-yl]carbonyl}glycine;
N-{[5-(methylamino)-1H-benzimidazol-4-yl]carbonyl}glycine;
N-{[6-bromo-5-(methyloxy)-1-(phenylmethyl)-1H-benzimidazol-4-yl]carbonyl}glycine;
N-{[6-bromo-5-hydroxy-1-(phenylmethyl)-1H-benzimidazol-4-yl]carbonyl}glycine;
N-[(5-ethyl-1H-benzimidazol-4-yl)carbonyl]glycine;
N-{[5-(dimethylamino)-1-(phenylmethyl)-1H-benzimidazol-4-yl]carbonyl}glycine;
N-{[5-(methyloxy)-1H-benzimidazol-4-yl]carbonyl}glycine;
N-{[1-[(4-bromophenyl)methyl]-5-(methyloxy)-1H-benzimidazol-4-yl]carbonyl}glycine;
N-{[1-(4-biphenylylmethyl)-5-(methyloxy)-1H-benzimidazol-4-yl]carbonyl}glycine;
N-[(5-(methyloxy)-1-{[4-(4-pyridinyl)phenyl]methyl}-1H-benzimidazol-4-yl)carbonyl]glycine;
N-[(5-(methyloxy)-1-{[4-(3-pyridinyl)phenyl]methyl}-1H-benzimidazol-4-yl)carbonyl]glycine; and
N-{[2-(aminocarbonyl)-5-(methyloxy)-1-(phenylmethyl)-1H-benzimidazol-4-yl]carbonyl}glycine;
or a pharmaceutically acceptable salt or solvate thereof.

5. A method for treating anemia in a mammal, which method comprises administering an effective amount of a compound of formula (I) or a salt or solvate thereof according to claim 1 to a mammalian suffering from anemia which can be treated by inhibiting HIF prolyl hydroxylases.

6. A pharmaceutical composition comprising a compound of formula (I) or a salt, solvate, according to claim 1 and one or more of pharmaceutically acceptable carriers, diluents and excipients.

7. A process for preparing a compound of formula (I) 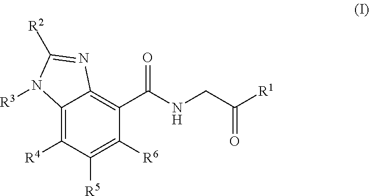
wherein R¹, R², R³, R⁴, R⁵, and R⁶ are the same as defined above for formula (I), the process comprising treating a compound of formula A: 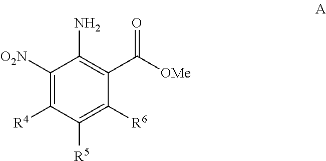
wherein R⁴, R⁵, and R⁶ are the same as for those groups in formula (I), in a hydrogen atmosphere with an appropriate catalyst, such as palladium on charcoal, in an appropriate solvent, such as ethyl acetate, followed by addition of an appropriately substituted orthoester, such as trimethyl orthoformate, neat or in an appropriate solvent, such as methanol, along with an appropriate acid, such as anhydrous hydrochloric acid in 1,4-dioxane or diethyl ether, to form a compound of formula B: 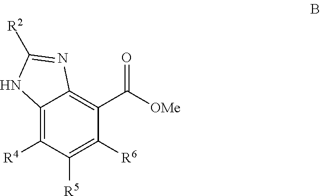
wherein R², R⁴, R⁵, and R⁶ are the same as for those groups in formula (I), which may then be deprotonated with an appropriate base, such as sodium hydride, in an appropriate solvent, such as tetrahydrofuran or N,N-dimethylformamide, and reacted with an appropriate alkylating agent, such as benzyl bromide or 2-chlorobenzyl bromide, followed by ester hydrolysis with an appropriate base, such as sodium hydroxide, in an appropriate solvent, such as tetrahydrofuran/methanol, to form a compound of formula C: 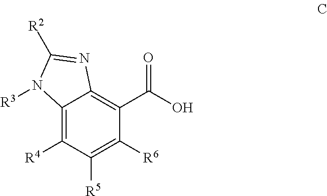
wherein R², R³, R⁴, R⁵, and R⁶ are the same as for those groups in formula (I), which is then coupled with an appropriate glycine ester, such as glycine ethyl ester hydrochloride, and an appropriate base, such as triethylamine or diisopropylethylamine, and an appropriate coupling reagent, such as HATU or PyBOP, in an appropriate solvent, such as N,N-dimethylformamide, followed by ester hydrolysis with an appropriate base, such as sodium hydroxide, in an appropriate solvent, such as tetrahydrofuran/methanol, to form a compound of formula (I) where R¹ is —OH.
